(19) United States
(12) Patent Application Publication
(10) Pub. No.: US 2025/0270561 A1
(43) Pub. Date: Aug. 28, 2025

(54) FUNCTIONAL NUCLEIC ACID MOLECULES

(71) Applicants: TRANSINE THERAPEUTICS LIMITED, Cambridge (GB); SCUOLA INTERNAZIONALE SUPERIORE DI STUDI, Trieste (IT)

(72) Inventors: Stefano GUSTINCICH, Genova (IT); Bianca PIERATTINI, Genova (IT); Carlotta BON, Cambridge (GB); Laura GRASSO, Cambridge (GB); Marc WATSON, Cambridge (GB)

(73) Assignees: TRANSINE THERAPEUTICS LIMITED, Cambridge, EN (GB); SCUOLA INTERNAZIONALE SUPERIORE DI STUDI AVANZATI, Trieste (IT)

(21) Appl. No.: 18/246,046

(22) PCT Filed: Oct. 8, 2021
(86) PCT No.: PCT/GB2021/052607

§ 371 Date: Feb. 21, 2024

(30) Foreign Application Priority Data
Oct. 8, 2020 (GB) 2015997.6
Dec. 8, 2020 (GB) 2019325.6

(51) Int. Cl. C12N 15/113; A61K 38/46; A61P 27/02

(52) U.S. Cl. CPC C12N 15/1137; A61K 38/46; A61P 27/02; C12Y 306/05005; C12N 2310/11; C12N 2310/532

(57) ABSTRACT

The present invention relates to functional nucleic acid molecules for use in upregulating OPA1 expression. The functional nucleic acid molecules typically comprise at least one target binding sequence reverse complementary to an OPA1 mRNA sequence and at least one regulatory sequence which comprises a SINE B2 element or an internal ribosome entry site (IRES) sequence. Also described are therapeutic methods of using the functional nucleic acids.

### FIELD OF THE INVENTION

The present invention relates to functional nucleic acid molecules for use in upregulating OPA1 expression.

### REFERENCE TO A SEQUENCE LISTING

The instant application contains a Sequence Listing that has been submitted in ASCII format via PatentCenter and is hereby incorporated by reference in its entirety. Said ASCII copy, created on Feb. 14, 2024, is named 2023_08_seq_listing_37686.04003_ST25.txt and is 35,407 bytes in size.

### BACKGROUND OF THE INVENTION

Autosomal Dominant Optic Atrophy (ADOA) is the most common inherited optic neuropathy, caused, in 75% of cases, by heterozygous mutations in OPA1 gene. ADOA is an early-onset autosomal dominant haploinsufficient disorder, with a prevalence ranging from 1:12000 to 1:50000 births and characterized by degeneration of the retinal ganglion cells that leads to optic nerve atrophy and blindness. Human OPA1 is a ubiquitously expressed dynamin-related GTPase protein with crucial functions in mitochondrial homeostasis, that localizes in the Inner Mitochondrial Membrane (IMM), reaching highest expression levels in brain, retina and heart.

Data indicate that both OPA1 under-expression, as seen in ADOA patients, and over-expression have deleterious consequences, with both of these alterations in OPA1 levels leading to elevations in apoptosis (Chen et al. (2009) Cardiovasc Res. 84(1): 91-9). In vivo, the data are complex. Whereas transgenic mice with mild OPA1 over-expression appear healthy and fertile and additionally exhibit protection against insults to specific tissue such as liver and brain, prolonged overexpression in the SV129 mouse strain was observed to increase incidence of spontaneous cancer and reduce lifespan (Varanita et al. (2015) Cell Metab. 21(6): 834-44). High expression of OPA1, and other mitochondrial proteins that promote fusion, is linked to cancerous cell proliferation, survival and invasion. OPA1 is highly expressed in lung adenocarcinoma cells and associated with cisplatin resistance and poor prognosis (Fang et al. (2012) Hum. Pathol. 43(1): 105-14).

A new class of long non-coding RNAs (lncRNAs), known as SINEUPs, were previously described to be able to selectively enhance their targets' translation. SINEUP activity relies on the combination of two domains: the overlapping region, or binding domain (BD), that confers specificity, and an embedded inverted SINE B2 element, or effector domain (ED), enhancing target mRNA translation. WO 2012/133947 and WO 2019/150346 disclose functional nucleic acid molecules including SINEUPs. Another class of lncRNAs that use effector domains comprising an internal ribosome entry site (IRES) sequence to provide trans-acting functional nucleic acid molecules are described in WO 2019/058304.

The aim of the invention is to provide the first gene-specific technology targeting OPA1 translation, particularly for use in treating ADOA.

### SUMMARY OF THE INVENTION

According to a first aspect, there is provided a functional nucleic acid molecule comprising:
at least one target binding sequence comprising a sequence reverse complementary to an OPA1 mRNA sequence; and
at least one regulatory sequence comprising an RNA comprising a SINE B2 element or a functionally active fragment of a SINE B2 element or an internal ribosome entry site (IRES) sequence or an IRES derived sequence.

According to a further aspect of the invention, there is provided a DNA molecule encoding the functional nucleic acid molecule defined herein. According to a further aspect of the invention, there is provided an expression vector comprising the functional nucleic acid molecule defined herein.

According to a further aspect of the invention, there is provided a composition comprising the functional nucleic acid molecule, the DNA molecule or the expression vector as defined herein.

According to a further aspect of the invention, there is provided a method for increasing the protein synthesis efficiency of OPA1 in a cell comprising administering the functional nucleic acid molecule, the DNA molecule, the expression vector or the composition as defined herein, to the cell.

According to a further aspect of the invention, there is provided a method of treating a disease associated with mitochondrial defects, such as ADOA, comprising administering a therapeutically effective amount of the functional nucleic acid molecule, the DNA molecule, the expression vector or the composition as defined herein.

According to a further aspect of the invention, there is provided a therapeutically effective amount of the functional nucleic acid molecule, the DNA molecule, the expression vector, or the composition for use in the manufacture of a medicament for the treatment of a disease associated with mitochondrial defects, such as ADOA.

### BRIEF DESCRIPTION OF THE FIGURES

FIG. 1: A schematic representation of SINEUP functional domains and of the human OPA1 gene with examples of the target binding domains of the functional nucleic acid according to the invention.

FIG. 2: HEK 293T cells were transfected with miniSINEUP deprived of BD (ABD) and miniSINEUP-OPA1 variants. (A) Whole cell lysates were analysed by western blotting with anti-OPA1 and anti-β-actin antibodies. One representative experiment is shown. Graphs show real-time PCR analysis of OPA1 mRNA and miniSINEUP RNA expression in transfected cells. Columns represent mean±S.E.M. of n≥3 independent experiments. (B) Average fold change of OPA1 protein levels. For each sample values are reported as two separate columns representing respectively the long (left) and the short (right) isoforms. Columns represent mean±S.E.M. of n≥4 independent experiments; ns, p>0.05; *p<0.05; **p<0.01; ***p<0.001; ****p<0.0001 (one-way ANOVA followed by Dunnett's post-test).

FIG. 3: Mouse Neruo2A cells were transfected with miniSINEUP deprived of BD (ABD) and miniSINEUP-OPA1 variants. (A) Whole cell lysates were analysed by western blotting with anti-OPA1 and anti-β-actin antibodies. One representative experiment is shown. Graphs show real-time PCR analysis of OPA1 mRNA and miniSINEUP RNA expression in transfected cells. Columns represent mean±S.E.M. of n≥3 independent experiments. (B) Average fold change of OPA1 protein levels. For each sample values are reported as two separate columns representing respectively the long (left) and the short (right) isoforms. Columns represent mean±S.E.M. of n≥3 independent experiments; ns, p>0.05; *p<0.05; **p<0.01; ***p<0.001; ****p<0.0001 (one-way ANOVA followed by Dunnett's post-test).

FIG. 4: Mouse Astrocyte cells were transfected with miniSINEUP deprived of BD (ΔBD) and miniSINEUP-OPA1 variants. (A) Whole cell lysates were analysed by western blotting with anti-OPA1 and anti-β-actin antibodies. One representative experiment is shown. Graphs show real-time PCR analysis of OPA1 mRNA and miniSINEUP RNA expression in transfected cells. Columns represent mean±S.E.M. of n≥3 independent experiments. (B) Average fold change of OPA1 protein levels. For each sample values are reported as two separate columns representing respectively the long (left) and the short (right) isoforms. Columns represent mean±S.E.M. of n≥3 independent experiments; ns, p>0.05; *p<0.05; **p<0.01; ***p<0.001; ****p<0.0001 (one-way ANOVA followed by Dunnett's post-test).

FIG. 5: HEK293T cells were transfected with miniSINEUP deprived of BD (ΔBD), miniSINEUP-OPA1 (−14/+4-M1-AUG) and microSINEUP-OPA1 (−14/+4-M1-AUG) variant. (A) Whole cell lysates were analysed by western blotting with anti-OPA1 and anti-β-actin antibodies. One representative experiment is shown. Graph shows real-time PCR analysis of OPA1 mRNA and miniSINEUP RNA expression in transfected cells. Columns represent mean±S.E.M. of n≥3 independent experiments. (B) Average fold change of OPA1 protein levels. For each sample values are reported as two separate columns representing respectively the long (left) and the short (right) isoforms. Columns represent mean±S.E.M. of n=4 independent experiments; ns, p>0.05; *p<0.05; **p<0.01; ***p<0.001; ****p<0.0001 (one-way ANOVA followed by Dunnett's post-test).

FIG. 6: HEK 293T cells were transfected with miniSINEUP lacking a binding domain (ΔBD) and nano2SINEUP-OPA1 (−14/+4-M1-AUG). (A) Whole cell lysates were analysed by western blotting with anti-OPA1 and anti-β-actin antibodies. One representative experiment is shown. (B) Real-time PCR analysis of OPA1 mRNA (left panel) and nano2SINEUP RNA expression (right panel). NT=non-transfected.

FIG. 7: miniSINEUPs in two different vector backbones showing OPA1-nanoluc luminescence in vitro.

FIG. 8: Results of synthetic nano2SINEUPs on OPA1 protein and mRNA levels. (A) Whole cell lysates were analysed by Western blotting with anti-OPA1 and anti-β-actin antibodies (B) Average fold change of OPA1 protein levels. Real-time PCR analysis of OPA1 mRNA (C) and nano2SINEUP RNA (D).

### DETAILED DESCRIPTION

It is an object of the present invention to provide a functional nucleic acid molecule that increases expression of the OPA1 protein without overcoming physiological levels, targets OPA1 expression for all isoforms in a highly gene-specific manner and limits side effects.

The inventors have utilised SINEUP technology to develop and target OPA1 in order to increase the endogenous levels of all OPA1 protein isoforms in human, mouse and patient derived cell lines. By in vitro screening, OPA1-specific SINEUPs were shown to be able to increase selectively both human and murine OPA1 proteins. Importantly, SINEUPs-OPA1 increased OPA1 protein levels in the 2-fold range when expressed in ADOA patients-derived fibroblasts, showing an activity sufficient for functional rescue, without leading to negative side-effects associated with increasing OPA1 expression above physiological levels. Furthermore, the SINEUPs-OPA1 did not disrupt the ratio of long/short forms of the OPA1 protein which is important in order to rescue mitochondrial network morphology.

### Definitions

By “functional nucleic acid molecule” there is intended generally that the nucleic acid molecule is capable of enhancing the translation of a target mRNA of interest, in this particular case an OPA1 mRNA.

By “OPA1 mRNA sequence” there is intended an mRNA sequence of any length of at least 10 nucleotides comprised in the mRNA of the corresponding OPA1 gene. Alternative splicing of the OPA1 transcript leads to the generation of eight different isoforms. They share their 5′ UTR and are ubiquitously expressed. The resulting OPA1 proteins also undergo cleavage to generate both long (I) and short(s) OPA1 forms. The OPA1 gene sequence is known in the art, for example see Gene ID: 4976 or Ensembl ID: ENSG00000198836.

The OPA1 gene encodes the OPA1 Mitochondrial Dynamin Like GTPase (also known as Dynamin-Like 120 KDa Protein, Mitochondrial) and is referred to herein as the “OPA1 protein”. The OPA1 protein sequence is known in the art, for example see UniProt ID: 060313.

The term “SINE” (Short Interspersed Nuclear Element) may be referred to as a non-LTR (long terminal repeat) retrotransposon, and is an interspersed repetitive sequence (a) which encodes a protein having neither reverse-transcription activity nor endonuclease activity or the like and (b) whose complete or incomplete copy sequences exist abundantly in genomes of living organisms.

The term “SINE B2 element” is defined in WO 2012/133947, where specific examples are also provided (see table starting on page 69 of the PCT publication). The term is intended to encompass both SINE B2 elements in direct orientation and in inverted orientation relative to the 5′ to 3′ orientation of the functional nucleic acid molecule. SINE B2 elements may be identified, for example, using programs like RepeatMask as published (Bedell et al. Bioinformatics. 2000 November; 16(11): 1040-1. MaskerAid: a performance enhancement to RepeatMasker). A sequence may be recognizable as a SINE B2 element by returning a hit in a Repbase database with respect to a consensus sequence of a SINE B2, with a Smith-Waterman (SW) score of over 225, which is the default cutoff in the RepeatMasker program. Generally a SINE B2 element is not less than 20 bp and not more than 400 bp. Preferably, the SINE B2 is derived from tRNA.

By the term “functionally active fragment of a SINE B2 element” there is intended a portion of sequence of a SINE B2 element that retains protein translation enhancing efficiency. This term also includes sequences which are mutated in one or more nucleotides with respect to the wild-type sequences, but retain protein translation enhancing efficiency. The term is intended to encompass both SINE B2 elements in direct orientation and in inverted orientation relative to the 5′ to 3′ orientation of the functional nucleic acid molecule.

The terms “internal ribosome entry site (IRES) sequence” and “internal ribosome entry site (IRES) derived sequence” are defined in WO 2019/058304. IRES sequences recruit the 40S ribosomal subunit and promote cap-independent translation of a subset of protein coding mRNAs. IRES sequences are generally found in the 5′ untranslated region of cellular mRNAs coding for stress-response genes, thus stimulating their translation in cis. It will be understood by the term “IRES derived sequence” there is intended a sequence of nucleic acid with a homology to an IRES sequence so as to retain the functional activity thereof, i.e. a translation enhancing activity. In particular, the IRES derived sequence can be obtained from a naturally occurring IRES sequence by genetic engineering or chemical modification, e.g. by isolating a specific sequence of the IRES sequence which remains functional, or mutating/deleting/introducing one or more nucleotides in the IRES sequence, or replacing one or more nucleotides in the IRES sequence with structurally modified nucleotides or analogs. More in particular, the skilled in the art would know that an IRES derived sequence is a nucleotide sequence capable of promoting translation of a second cistron in a bicistronic construct. Typically, a dual luciferase (Firefly luciferase, Renilla Luciferase) encoding plasmid is used for experimental tests. A major database exists, namely IRESite, for the annotation of nucleotide sequences that have been experimentally validated as IRES, using dual reporter or bicistronic assays (http://iresite.org/IRESite_web.php). Within the IRESite, a web-based tool is available to search for sequence-based and structure-based similarities between a query sequence of interest and the entirety of annotated and experimentally validated IRES sequences within the database. The output of the program is a probability score for any nucleotide sequence to be able to act as IRES in a validation experiment with bicistronic constructs. Additional sequence-based and structure-based web-based browsing tools are available to suggest, with a numerical predicting value, the IRES activity potentials of any given nucleotide sequence (http://rna.informatik.uni-freiburg.de/; http://regrna.mbc.nctu.edu.tw/index1.php).

By the term “miniSINEUP” there is intended a nucleic acid molecule comprising (or consisting of) a binding domain (i.e. a complementary sequence to target mRNA), optionally a spacer sequence, and any SINE or SINE-derived sequence or IRES or IRES-derived sequence as the effector domain (Zucchelli et al., Front Cell Neurosci., 9:174, 2015).

By the term “microSINEUP” there is intended a nucleic acid molecule comprising (or consisting of) a binding domain (i.e. a complementary sequence to target mRNA), optionally a spacer sequence, and a functionally active fragment of the SINE or SINE-derived sequence or IRES-derived sequence. For example, the functionally active fragment may be a 77 bp sequence corresponding to nucleotides 44 to 120 of the 167 bp SINE B2 element in ΔS Uchl1.

Polypeptide or polynucleotide sequences are said to be the same as or “identical” to other polypeptide or polynucleotide sequences, if they share 100% sequence identity over their entire length. Residues in sequences are numbered from left to right, i.e. from N- to C-terminus for polypeptides; from 5′ to 3′ terminus for polynucleotides.

For the purposes of comparing two closely-related polynucleotide sequences, the “% sequence identity” between a first nucleotide sequence and a second nucleotide sequence may be calculated using NCBI BLAST, using standard settings for nucleotide sequences (BLASTN). For the purposes of comparing two closely-related polypeptide sequences, the “% sequence identity” between a first polypeptide sequence and a second polypeptide sequence may be calculated using NCBI BLAST, using standard settings for polypeptide sequences (BLASTP). A “difference” between sequences refers to an insertion, deletion or substitution of a single nucleotide in a position of the second sequence, compared to the first sequence. Two sequences can contain one, two or more such differences. Insertions, deletions or substitutions in a second sequence which is otherwise identical (100% sequence identity) to a first sequence result in reduced % sequence identity.

### Functional Nucleic Acid Molecules

A functional nucleic acid molecule of the present invention comprises at least one target binding sequence comprising a sequence reverse complementary to an OPA1 mRNA sequence and at least one regulatory sequence comprising an RNA comprising a SINE B2 element or a functionally active fragment of a SINE B2 element or an internal ribosome entry site (IRES) sequence or an IRES derived sequence.

### Regulatory Sequences

The regulatory sequence has protein translation enhancing efficiency. The increase of the protein translation efficiency indicates that the efficiency is increased as compared to a case where the functional nucleic acid molecule according to the present invention is not present in a system. In one embodiment, expression of the protein encoded by the target mRNA is increased by at least 1.5 fold, such as at least 2 fold. In a further embodiment, expression of the protein encoded by the target mRNA is increased between 1.5 to 3 fold, such as between 1.6 and 2.2 fold. It is envisaged that increasing protein expression within these ranges will allow the treatment of diseases associated with mitochondrial defects, such as ADOA, without leading to negative side-effects associated with increasing OPA1 expression above physiological levels.

In one embodiment, the regulatory sequence is located 3′ of the target binding sequence. The regulatory sequence may be in a direct or inverted orientation relative to the 5′ to 3′ orientation of the functional nucleic acid molecule. Reference to “direct” refers to the situation in which the regulatory sequence is embedded (inserted) with the same 5′ to 3′ orientation as the functional nucleic acid molecule. Instead, “inverted” refers to the situation in which the regulatory sequence is 3′ to 5′ oriented relative to the functional nucleic acid molecule.

Preferably, the at least one regulatory sequence comprises a sequence with at least about 80% sequence identity, at least about 85% sequence identity, at least about 90% sequence identity, at least about 91% sequence identity, at least about 92% sequence identity, at least about 93% sequence identity, at least about 94% sequence identity, preferably at least about 95% sequence identity, at least about 96% sequence identity, at least about 97% sequence identity, at least about 98% sequence identity, at least about 99% sequence identity, more preferably 100% sequence identity with a sequence selected from the group consisting of SEQ ID NO: 1-69. In one embodiment, the at least one regulatory sequence consists of a sequence with at least about 80% sequence identity, at least about 85% sequence identity, at least about 90% sequence identity, at least about 91% sequence identity, at least about 92% sequence identity, at least about 93% sequence identity, at least about 94% sequence identity, preferably at least about 95% sequence identity, at least about 96% sequence identity, at least about 97% sequence identity, at least about 98% sequence identity, at least about 99% sequence identity, more preferably 100% sequence identity with a sequence selected from the group consisting of SEQ ID NO: 1-69.

In one embodiment, the regulatory sequence comprises a SINE B2 element or a functionally active fragment of a SINE B2 element. The SINE B2 element is preferably in an inverted orientation relative to the 5′ to 3′ orientation of the functional nucleic acid molecule, i.e. an inverted SINE B2 element. As mentioned in the definitions section, inverted SINE B2 elements are disclosed and exemplified in WO 2012/133947.

In one embodiment, the at least one regulatory sequence comprises a sequence with at least about 80% sequence identity, at least about 85% sequence identity, at least about 90% sequence identity, at least about 91% sequence identity, at least about 92% sequence identity, at least about 93% sequence identity, at least about 94% sequence identity, preferably at least about 95% sequence identity, at least about 96% sequence identity, at least about 97% sequence identity, at least about 98% sequence identity, at least about 99% sequence identity, more preferably 100% sequence identity with a sequence selected from the group consisting of SEQ ID NO: 1-51.

SEQ ID NO: 1 (the 167 nucleotide variant of the inverted SINE B2 element in ΔS Uchl1) and SEQ ID NO: 2 (the 77 nucleotide variant of the inverted SINE B2 element in ΔS Uchl1 that includes nucleotides 44 to 120), as well as sequences with percentage identity to these sequences, are particularly preferred.

Other inverted SINE B2 elements and functionally active fragments of inverted SINE B2 elements are SEQ ID NO: 3-51. Experimental data showing the protein translation enhancing efficiency of these sequences is not explicitly shown in the present patent application, but is disclosed in a previous patent application in the name of the same applicant. SEQ ID NO: 3-51 can therefore also be used as regulatory sequences in molecules according to the present invention.

SEQ ID NO: 3-6, 8-11, 18, and 43-51 are functionally active fragments of inverted SINE B2 transposable element derived from AS Uchl1. The use of functional fragments reduces the size of the regulatory sequence which is advantageous if used in an expression vector (e.g. viral vectors which may be size-limited) because this provides more space for the target sequence and/or expression elements.

SEQ ID NO: 7 is a full length 183 nucleotide inverted SINE B2 transposable element derived from AS Uchl1. SEQ ID NO: 12-17, 19, 20, and 39-42 are mutated functionally active fragments of inverted SINE B2 transposable element derived from AS Uchl1.

SEQ ID NO: 21-25, and 28-38 are different SINE B2 transposable elements. SEQ ID NO: 26 and 27 are sequences in which multiple inverted SINE B2 transposable element have been inserted.

Alternatively, the regulatory sequence comprises an IRES sequence or an IRES derived sequence. Therefore, in one embodiment, the regulatory sequence comprises an IRES sequence or an IRES derived sequence. Said sequence enhances translation of the target mRNA sequence.

Several IRESs having sequences ranging from 48 to 576 nucleotides have been tested with success, e.g. human Hepatitis C Virus (HCV) IRESs (e.g. SEQ ID NO: 52 and 53), human poliovirus IRESs (e.g. SEQ ID NO: 54 and 55), human encephalomyocarditis (EMCV) virus (e.g. SEQ ID NO: 56 and 57), human cricket paralysis (CrPV) virus (e.g. SEQ ID NO: 58 and 59), human Apaf-1 (e.g. SEQ ID NO: 60 and 61), human ELG-1 (e.g. SEQ ID NO: 62 and 63), human c-MYC (e.g. SEQ ID NO: 64-67), and human dystrophin (DMD) (e.g. SEQ ID NO: 68 and 69).

Such sequences have been disclosed, defined and exemplified in WO 2019/058304. Preferably, such sequences have at least about 80% sequence identity, at least about 85% sequence identity, at least about 90% sequence identity, at least about 91% sequence identity, at least about 92% sequence identity, at least about 93% sequence identity, at least about 94% sequence identity, preferably at least about 95% sequence identity, at least about 96% sequence identity, at least about 97% sequence identity, at least about 98% sequence identity, at least about 99% sequence identity, more preferably 100% sequence identity to any of SEQ ID NO: 52-69.

### Target Binding Sequences

Human OPA1 is a dynamin-related GTPase protein, encoded in locus 3q28, that localizes in the Inner Mitochondrial Membrane (IMM) and is ubiquitously expressed, reaching highest expression levels in the brain, retina and heart. The gene is constituted by 30 exons and the protein itself is translated into eight different isoforms depending on alternative mRNA splicing and processing. In the IMM, the mitochondrial targeting sequence (MTS) is cleaved on the first cleavage site to generate the long transmembrane forms. Isoform 4, 6, 7 and 8 also contain a second cleavage site, encoded in exon 5b, that leads to their further processing into the short forms of the protein. OPA1 has primary functions in mitochondrial homeostasis. Together with the mitofusins MFN1 and MFN2 it promotes fusion of mitochondria, a process associated with increased respiratory efficiency, and it contributes to mitochondrial DNA (mtDNA) maintenance. OPA1 protein polymerization also preserves cristae morphogenesis, facilitating activity of the respiratory super-complexes. It has a main role in controlling apoptotic process as it is fundamental for the compartmentalization of cytochrome C, whose uncontrolled release leads to cell death. Any of the eight OPA1 isoforms can support its three essential functions (energetics, structural and mtDNA maintenance), but a balance between long and short isoforms seems to be a crucial requirement for a full recovery of the mitochondrial network. The complete rescue of mitochondrial network morphology therefore requires a balance of long and short forms of at least two isoforms. The data presented herein shows that the OPA1-SINEUP is a unique tool to target all OPA1 transcripts at the same time restoring the correct physiological ratio and processing to the I/s forms in the regular physiological manner. This differs from alternative therapeutic approaches which may favour the expression of a single specific transcript/isoform and potentially disrupts the physiological ratio of OPA1 isoforms and the ratio of I/s OPA1 protein.

In WO 2012/133947 it was already shown that the target binding sequence needs to have only about 60% similarity with a sequence reverse complementary to the target mRNA. As a matter of fact, the target binding sequence can even display a large number of mismatches and retain activity.

In one embodiment, the target binding sequence comprises a sequence reverse complementary to a portion of the OPA1 mRNA sequence that is common to all OPA1 isoforms. By maintaining the reciprocal levels of all OPA1 isoforms, the functional nucleic acid molecule are able to induce the best molecular pattern of expression to restore physiological homeostasis. This is not possible with a more conventional gene therapy approaches when only one isoform can be ectopically expressed leading to isoform imbalance.

The target binding sequence comprises a sequence which is sufficient in length to bind to the OPA1 mRNA transcript. Therefore, the target binding sequence may be at least about 10 nucleotides long, such as at least about 14 nucleotides long, such as at least about 15 nucleotides long, such as at least about 16 nucleotides long, such as at least about 17 nucleotides long, such as least about 18 nucleotides long. Furthermore, the target binding sequence may be less than about 250 nucleotides long, preferably less than about 200 nucleotides long, less than about 150 nucleotides long, less than about 140 nucleotides long, less than about 130 nucleotides long, less than about 120 nucleotides long, less than about 110 nucleotides long less than about 100 nucleotides long, less than about 90 nucleotides long, less than about 80 nucleotides long, less than about 70 nucleotides long, less than about 60 nucleotides long or less than about 50 nucleotides long. In one embodiment, the target binding sequence is between about 4 and about 50 nucleotides in length, such as between about 18 and about 44 nucleotides long.

The target binding sequence may be designed to hybridise with the 5′-untranslated region (5′ UTR) of the OPA1 mRNA sequence. In one embodiment, the sequence is reverse complementary to 0 to 50 nucleotides, such as 0 to 41, 0 to 40, 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, or 0 to 6 nucleotides of the 5′ UTR. Alternatively, or in combination, the target binding sequence may be designed to hybridise to the coding sequence (CDS) of the OPA1 mRNA sequence. In one embodiment, the sequence is reverse complementary to 0 to 40 nucleotides, such as 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4 or 0 nucleotides of the CDS.

The target binding sequence may be designed to hybridise to a region upstream of an AUG site (start codon), such as a start codon within the CDS, of the OPA1 mRNA sequence. In one embodiment, the sequence is reverse complementary to 0 to 80 nucleotides, such as 0 to 70, 0 to 60, 0 to 50, 0 to 40, 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, or 0 to 9 nucleotides of the AUG site. Alternatively, or in combination, the target binding sequence may be designed to hybridise to the OPA1 mRNA sequence downstream of said AUG site. In one embodiment, the sequence is reverse complementary to 0 to 40 nucleotides, such as 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4 or 0 nucleotides of the OPA1 mRNA sequence downstream of said AUG site.

Preferably, the target binding sequence is at least 10 nucleotides long and comprises, from 3′ to 5′:
1) a sequence reverse complementary to 0 to 50 nucleotides of the 5′ UTR and 0 to 40 nucleotides of the CDS of the OPA1 mRNA sequence; or
2) a sequence reverse complementary to 0 to 80 nucleotides of the region upstream of an AUG site (start codon) of the OPA1 mRNA and 0 to 40 nucleotides of the OPA1 mRNA sequence downstream of said AUG site.

In case 1) the coding sequence starts on the first AUG site (M1) of the mRNA. In case 2), the preferred AUG site is that corresponding to an internal start codon, such as methionine 125 (M125) in exon 3. In the context of referencing a sequence reverse complementary to a region in the 5′ UTR and the CDS, this is preferably anchored around the AUG site, i.e. the region in the 5′ UTR is directly upstream of the AUG site of the target mRNA. For example, reference to a target binding sequence that is “−40/+4 of M1” refers to a target binding sequence that is reverse complementary to the 40 nucleotides within the 5′ UTR upstream of the AUG site (−40) and the 4 nucleotides within the CDS downstream of the AUG site (+4).

In accordance with conventional numbering, the nucleotides of the 5′UTR sequence are numbered sequentially using decreasing negative numbers approaching the AUG site on the target mRNA (e.g. −3, −2, −1). The nucleotides of the CDS sequence are numbered sequentially using increasing positive numbers (e.g. +1, +2, +3) from the AUG site, such that the A of the AUG site is numbered +1. The region bridging the 5′UTR and the CDS will therefore be numbered −3, −2, −1, +1, +2, +3, with the A of the AUG site numbered +1.

More preferably, the at least one target binding sequence is at least 14 nucleotides long and comprises, from 3′ to 5′:
a sequence reverse complementary to 0 to 40 (preferably 0 to 21, more preferably 0 to 14) nucleotides of the 5′ UTR and 0 to 32 (preferably 0 to 4, more preferably 0) nucleotides of the CDS of the OPA1 mRNA sequence; or
a sequence reverse complementary to 0 to 70 (preferably 0 to 40) nucleotides of the region upstream of an AUG site (start codon) of the OPA1 mRNA and 0 to 4 (preferably 0) nucleotides of the CDS of the OPA1 mRNA sequence downstream of said AUG site.

In a particular embodiment, the target binding sequence is 18 nucleotides long and comprises, from 3′ to 5′ a sequence reverse complementary to 14 nucleotides of the of the 5′ UTR and 4 nucleotides of the CDS of the OPA1 mRNA sequence. For example, the target binding sequence may comprise a sequence encoded by the DNA sequence of SEQ ID NO: 82 (i.e. −14/+4 of M1).

In a further particular embodiment, the target binding sequence is 15 nucleotides long and comprises, from 3′ to 5′ a sequence reverse complementary to 6 nucleotides of the of the 5′ UTR and 9 nucleotides of the CDS of the OPA1 mRNA sequence. For example, the target binding sequence may comprise a sequence encoded by the DNA sequence of SEQ ID NO: 83 (i.e. −6/+9 of M1).

In another particular embodiment, the target binding sequence is 12 nucleotides long and comprises, from 3′ to 5′ a sequence reverse complementary to 41 nucleotides of the of the 5′ UTR and 30 nucleotides of the CDS of the OPA1 mRNA sequence. For example, the target binding sequence may comprise a sequence encoded by the DNA sequence of SEQ ID NO: 84 (i.e. −41/+30 of M1).

In another particular embodiment, the target binding sequence is 12 nucleotides long and comprises, from 3′ to 5′ a sequence reverse complementary to 41 nucleotides of the of the 5′ UTR and 30 nucleotides of the CDS of the OPA1 mRNA sequence. For example, the target binding sequence may comprise a sequence encoded by the DNA sequence of SEQ ID NO: 93 (i.e. −41/−30 of M1).

In a further embodiment, the target binding sequence is 14 nucleotides long and comprises, from 3′ to 5′ a sequence reverse complementary to a region between nucleotides 97 and 84 of the of the 5′ UTR of the OPA1 mRNA sequence. For example, the target binding sequence may comprise a sequence encoded by the DNA sequence of SEQ ID NO: 85 (i.e. −97/−87 of M1).

In a yet further embodiment, the target binding sequence is 18 nucleotides long and comprises, from 3′ to 5′ a sequence reverse complementary to 18 nucleotides of the of the 5′ UTR and 0 nucleotides of the CDS of the OPA1 mRNA sequence. For example, the target binding sequence may comprise a sequence encoded by the DNA sequence of SEQ ID NO: 86 (i.e. −18/−1 of M1).

In another embodiment, the target binding sequence is 22 nucleotides long and comprises, from 3′ to 5′ a sequence reverse complementary to 18 nucleotides of the of the 5′ UTR and 4 nucleotides of the CDS of the OPA1 mRNA sequence. For example, the target binding sequence may comprise a sequence encoded by the DNA sequence of SEQ ID NO: 87 (i.e. −18/+4 of M1).

In a further embodiment, the target binding sequence is 14 nucleotides long and comprises, from 3′ to 5′ a sequence reverse complementary to 14 nucleotides of the of the 5′ UTR and 0 nucleotides of the CDS of the OPA1 mRNA sequence. For example, the target binding sequence may comprise a sequence encoded by the DNA sequence of SEQ ID NO: 88 (i.e. −14/−1 of M1).

In one particular embodiment, the target binding sequence is 17 nucleotides long and comprises, from 3′ to 5′ a sequence reverse complementary to 9 nucleotides of the region upstream of an AUG site (start codon) of the OPA1 mRNA and 8 nucleotides of the CDS of the OPA1 mRNA sequence downstream of said AUG site. For example, the target binding sequence may comprise a sequence encoded by the DNA sequence of SEQ ID NO: 89 (i.e. −9/+8 of M125).

In another embodiment, the target binding sequence is 18 nucleotides long and comprises, from 3′ to 5′ a sequence reverse complementary to 18 nucleotides of the region upstream of an AUG site (start codon) of the OPA1 mRNA and 0 nucleotides of the CDS of the OPA1 mRNA sequence downstream of said AUG site. For example, the target binding sequence may comprise a sequence encoded by the DNA sequence of SEQ ID NO: 90 (i.e. −18/−1 of M125).

In a further embodiment, the target binding sequence is 22 nucleotides long and comprises, from 3′ to 5′ a sequence reverse complementary to 18 nucleotides of the region upstream of an AUG site (start codon) of the OPA1 mRNA and 4 nucleotides of the CDS of the OPA1 mRNA sequence downstream of said AUG site. For example, the target binding sequence may comprise a sequence encoded by the DNA sequence of SEQ ID NO: 91 (i.e. −18/+4 of M125).

In a yet further embodiment, the target binding sequence is 14 nucleotides long and comprises, from 3′ to 5′ a sequence reverse complementary to 14 nucleotides of the region upstream of an AUG site (start codon) of the OPA1 mRNA and 0 nucleotides of the CDS of the OPA1 mRNA sequence downstream of said AUG site. For example, the target binding sequence may comprise a sequence encoded by the DNA sequence of SEQ ID NO: 92 (i.e. −14/−1 of M125).

In a yet further embodiment, the target binding sequence is 44 nucleotides long and comprises, from 3′ to 5′ a sequence reverse complementary to 40 nucleotides of the region upstream of an AUG site (start codon) of the OPA1 mRNA and 4 nucleotides of the CDS of the OPA1 mRNA sequence downstream of said AUG site. For example, the target binding sequence may comprise a sequence encoded by the DNA sequence of SEQ ID NO: 94 (−40/+4 of M1).

In a yet further embodiment, the target binding sequence is 44 nucleotides long and comprises, from 3′ to 5′ a sequence reverse complementary to 40 nucleotides of the region upstream of an AUG site (start codon) of the OPA1 mRNA and 4 nucleotides of the CDS of the OPA1 mRNA sequence downstream of said AUG site. For example, the target binding sequence may comprise a sequence encoded by the DNA sequence of SEQ ID NO: 95 (−40/+4 of M2).

Thus, in some embodiments, the target binding sequence comprises a sequence encoded by a DNA sequence with at least about 75% sequence identity, at least about 80% sequence identity, at least about 85% sequence identity, preferably at least about 90% sequence identity, at least about 91% sequence identity, at least about 92% sequence identity, at least about 93% sequence identity, at least about 94% sequence identity, more preferably at least about 95% sequence identity, at least about 96% sequence identity, at least about 97% sequence identity, at least about 98% sequence identity, at least about 99% sequence identity, even more preferably 100% sequence identity to any of SEQ ID NOs: 82-95, preferably SEQ ID NOs: 82-84 or 89. In a further embodiment, the target binding sequence consists of a sequence encoded by the DNA sequence with at least about 75% sequence identity, at least about 80% sequence identity, at least about 85% sequence identity, preferably at least 90% sequence identity, at least about 91% sequence identity, at least about 92% sequence identity, at least about 93% sequence identity, at least about 94% sequence identity more preferably at least about 95% sequence identity, at least about 96% sequence identity, at least about 97% sequence identity, at least about 98% sequence identity, at least about 99% sequence identity, even more preferably 100% sequence identity to any of SEQ ID NOs: 82-95, preferably SEQ ID NOs: 82-84 or 89.

In one embodiment, the functional nucleic acid molecule comprises a sequence with at least about 75% sequence identity, at least about 80% sequence identity, at least about 85% sequence identity preferably at least about 90% sequence identity, at least about 91% sequence identity, at least about 92% sequence identity, at least about 93% sequence identity, at least about 94% sequence identity, more preferably at least about 95% sequence identity, at least about 96% sequence identity, at least about 97% sequence identity, at least about 98% sequence identity, at least about 99% sequence identity even more preferably 100% sequence identity to any of SEQ ID NO: 70-79, preferably SEQ ID NOs: 70-74, 78-79. In a further embodiment, the functional nucleic acid molecule consists of a sequence with at least about 75% sequence identity, at least about 80% sequence identity, at least about 85% sequence identity, preferably at least about 90% sequence identity, at least about 91% sequence identity, at least about 92% sequence identity, at least about 93% sequence identity, at least about 94% sequence identity, more preferably at least about 95% sequence identity, at least about 96% sequence identity, at least about 97% sequence identity, at least about 98% sequence identity, at least about 99% sequence identity, even more preferably 100% sequence identity to any of SEQ ID NO: 70-79, preferably SEQ ID NOs: 70-74, 78-79.

More specifically, SEQ ID NOs: 70-74, 78-79 relate to functional nucleic acid molecules directed to human OPA1 isoforms, whereas SEQ ID NO: 75-77 relate to functional nucleic acid molecules directed to mouse OPA1 isoforms. Furthermore, SEQ ID NOs: 70-77 comprise the ‘mini’ inverted SINE B2 element in AS Uchl1 (167 nucleotides), SEQ ID NO: 78 comprises the ‘micro’ inverted SINE B2 element in AS Uchl1 (i.e. nucleotides 44-120 of inverted SINE B2 transposable element derived from AS Uchl1) and SEQ ID NO: 79 comprises the ‘nano’ inverted SINE B2 element in AS Uchl1 (i.e. nucleotides 64-92 of inverted SINE B2 transposable element derived from AS Uchl1). The differences between the miniSINEUP sequences is by virtue of the targeting binding sequence and/or the spacer/linker sequence as described herein.

As will be appreciated from the disclosures herein and the sequence identity of human OPA1 isoforms to those of, for example, mouse and rhesus macaque (Macaca mulatta), the target binding sequences presented herein may have cross-reactivity with other species. For example, human OPA1 mRNA has 96.19% sequence identity across its entire length with Macaca mulatta OPA1 mRNA and 78.36% sequence identity with mouse OPA1 mRNA. Thus, in one embodiment, the target binding sequence comprising or consisting of any of the sequences encoded by the DNA sequence of SEQ ID NOs: 82-95 binds mouse OPA1 and/or Macaca mulatta OPA1, preferably Macaca mulatta OPA1.

In one embodiment, the functional nucleic acid molecules provided herein are chemically modified. The term “modification” or “chemical modification” refers to a structural change in, or on, the most common, natural ribonucleotides: adenosine, guanosine, cytidine, or uridine ribonucleotides. Chemical modifications may be changes in or on a nucleobase (i.e. a chemical base modification), or in or on a sugar (i.e. a chemical sugar modification). The chemical modifications may be introduced co-transcriptionally (e.g. by substitution of one or more nucleotides with a modified nucleotide during synthesis), or post-transcriptionally (e.g. by the action of an enzyme).

Chemical modifications are known in the art, for example as described in The RNA Modification Database provided by The RNA Institute (https://mods.rna.albany.edu/mods/). Many modifications occur in nature, such as chemical modifications to natural transfer RNAs (tRNAs), which include, for example: 2′-O-Methyl (such as 2′-O-Methyladenosine, 2′-O-Methylguanosine and 2′-O-Methylpseudouridine), 1-Methyladenosine, 2-Methyladenosine, 1-Methylguanosine, 7-Methylguanosine, 2-Thiocytidine, 5-Methylcytidine, 5-Formylcytidine, Pseudouridine, Dihydrouridine, or the like.

### Structural Features

The functional nucleic acid molecule may comprise more than one regulatory sequence, which can be the same sequence repeated more than once, or a different regulatory sequence (i.e. a different SINE B2 element/functionally active fragment of a SINE B2 element/an IRES sequence/an IRES derived sequence).

The at least one target binding sequence and the at least one regulatory sequence are preferably connected by at least one spacer/linker sequence. SEQ ID NOs: 80 or 81 are non-limiting examples of the spacer/linker sequence that may be used. Fragments of these sequences are also contemplated.

The functional nucleic acid molecule of the present invention is preferably a circular molecule. This conformation leads to a much more stable molecule that is degraded with greater difficulty within the cell (exonucleases cannot degrade circular molecules) and therefore remains active for a longer time.

Furthermore, the functional nucleic acid molecule may optionally comprise a non-coding 3′ tail sequence, which e.g. includes restriction sites useful for cloning the molecule in appropriate plasmids.

In one embodiment, the functional nucleic acid molecule comprises a 3′-polyadenylation (polyA) tail. A “3′-polyA tail” refers to a long chain of adenine nucleotides added to the 3′-end of the transcription which provides stability to the RNA molecule and can promote translation.

In one embodiment the functional nucleic acid molecule comprises a 5′-cap. A “5′-cap” refers to an altered nucleotide at the 5′-end of the transcript which provides stability to the molecule, particularly from degradation from exonucleases, and can promote translation.

It should be noted that the functional nucleic acid molecules can enhance translation of the target gene of interest with no effects on mRNA quantities of the target gene. Therefore they can successfully be used as molecular tools to validate gene function in cells as well as to implement the pipelines of recombinant protein production.

### DNA Molecules and Vectors

According to a further aspect of the invention, there is provided a DNA molecule encoding any of the functional nucleic acid molecules disclosed herein. According to a further aspect of the invention, there is provided an expression vector comprising said DNA molecule.

Exemplary expression vectors are known in the art and may include, for example, plasmid vectors, viral vectors (for example adenovirus, adeno-associated virus, retrovirus or lentivirus vectors), phage vectors, cosmid vectors and the like. The choice of expression vector may be dependent upon the type of host cell to be used and the purpose of use. In particular the following plasmids have been used for efficient expression of functional nucleic acid molecules:

### Mammalian Expression Plasmids:

Plasmid Name: pCDNA3.1 (−)
Expression: CMV promoter
   BGH poly(A) terminator
Plasmid Name: pDUAL-eGFPΔ (modified from peGFP-C2)
Expression: H1 promoter
   BGH poly(A) terminator

### Viral Vectors:

Vector Name: pAAV
Virus: Adeno-Associated Virus
Expression: CAG promoter/CMV enhancer
   SV40 late poly(A) terminator
Vector Name: rcLV-TetOne-Puro
Virus: Lentivirus (3rd generation)
Expression: LTR-TREt (Tre-Tight) promoter (doxycycline-inducible expression)
   BGH poly(A) terminator
Vector Name: pLPCX-link
Virus: Retrovirus (3rd generation)
Expression: CMV

It should be noted that any promoter may be used in the vector and will work just as well as those mentioned above.

### Compositions and Medical Uses

The present invention also relates to compositions comprising the functional nucleic acid molecules, the DNA molecules and the expression vectors described herein. The composition may comprise components which enable delivery of said functional nucleic acid molecules by viral vectors (AAV, lentivirus and the like) and non-viral vectors (nanoparticles, lipid particles and the like).

The functional nucleic acid molecule of the invention may also be administered as naked or unpackaged RNA. Alternatively, the functional nucleic acid molecule may be administered as part of a composition, for example compositions comprising a suitable carrier. In certain embodiments, the carrier is selected based upon its ability to facilitate the transfection of a target cell with one or more functional nucleic acid molecules.

According to a further aspect of the invention, there is provided the functional nucleic acid molecule, DNA molecule, expression vector or the composition as defined herein for use as a medicament.

It will be understood that the functional nucleic acid molecules of the invention find use in increasing the level of OPA1 protein within a cell. OPA1 has primary functions in mitochondrial homeostasis therefore, according to a further aspect of the invention, there is provided the functional nucleic acid molecule, DNA molecule, expression vector or composition for use in the treatment of a disease-associated with mitochondrial defects.

The above said functional nucleic acid molecules, DNA molecules and/or compositions are used as medicaments, preferably for treating Autosomal Dominant Optic Atrophy (ADOA) and in particular promoting the recovery of disease-associated mitochondrial defects. Retinal ganglion cells (RGCs) expressing mutated OPA1 and RGC-specific OPA1 deficient mice have been shown to play a role in autophagy in ADOA pathogenesis (Zaninello et al. (2020) Nat. Comm. 11(1): 4029).

Therefore, in a further embodiment, the disease-associated with mitochondrial defects is ADOA. ADOA is the most common inherited optic neuropathy, caused in the 75% of cases by heterozygous mutations in OPA1 gene. The main symptoms of the disease are a bi-lateral degeneration of Retinal Ganglion Cells (RGCs) and optic nerve atrophy with possible muscular and neurodegenerative symptoms associated. Various forms of ADOA have been reported, such as ADOA plus, which also displays muscular defects and neurosensory deafness, and ADOAC, which leads to cataract too. Furthermore, intra-familial and inter-familial variations in severity of the disease among patients with the same mutation have been reported.

According to a further aspect of the invention, there is provided the use of the functional nucleic acid molecule (or DNA molecule, expression vector or composition) as defined herein for the manufacture of a medicament for the treatment of a disease-associated with mitochondrial defects, such as ADOA.

According to another aspect of the invention, there is provided a therapeutically effective amount of the functional nucleic acid molecule, the DNA molecule, the expression vector, or the composition defined herein for use in the manufacture of a medicament for the treatment of a disease associated with mitochondrial defects, such as ADOA.

In general, OPA1 is one of the main factors that control mitochondrial fusion, mitochondrial DNA (mtDNA) maintenance, bioenergetics, and cristae integrity. These cellular processes are targets of several diseases that can be potentially rescued by increase of OPA1 endogenous expression. In addition, OPA1 also controls apoptosis through cristae remodelling and cytochrome c release independently from mitochondrial fusion (Frezza et al. (2006) Cell 126(1): 177-89).

In addition to ADOA some reports propose that a mild increase of OPA1 protein expression may be therapeutic for additional diseases. For example, Civiletto et al. (2015) Cell Metab. 21(6): 845-854 showed that moderate OPA1 overexpression ameliorates the phenotype of two mitochondrial disease mouse models that had defects in the Ndufs4 or Cox15 genes. In humans, mutations in NDUFS4 are associated with early-onset fatal Leigh syndrome due to severe Complex I (CI) deficiency, while mutations in COX15 have been reported in children with severe isolated cardiomyopathy, encephalopathy, or cardioencephalomyopathy. As another example, Varanita et al. (2015) Cell Metabolism showed that Opa1^{tg} mice (a model overexpressing OPA1 about 1.5 fold—also see Cogliati et al. (2013) Cell 155(1): 160-171) are protected from muscular atrophy, myocardial infarction, less susceptible to Fas-induced liver damage, mitochondria are resistant to cristae remodelling and cytochrome C release. In contrast, large overexpression of OPA1 has been indicated to be toxic (Cipolat et al. (2004) PNAS 101(45): 15927-15932), therefore the methods provided herein are particularly well suited to treating diseases associated with OPA1 deficiencies because it is important to only increase expression to normal, physiological levels. It is envisaged that this will avoid unwanted side-effects which may be associated with a large increase in OPA1 expression above physiological levels.

Diseases associated with mitochondrial defects are well known in the art, for example as described in Gorman et al. (2016) Nat. Rev. Disease Primers, 2, 16080. They may be characterised by defects in oxidative phosphorylation due to mutations in nuclear or mitochondrial DNA that result in mutated/dysfunctional mitochondrial proteins.

Mitochondrial defects have been associated with neural disease and development. Therefore, in one embodiment, the disease-associated with mitochondrial defects is a neurological disease. Caglayan et al. (2020) iScience 23:101154, described genetically modified human embryonic and patient-derived induced pluripotent stem cells with OPA1 haploinsufficiency led to aberrant nuclear DNA methylation and significantly altered the transcriptional circuitry in neural progenitor cells (NPCs). In particular, OPA1+/−NPCs could not develop into GABAergic interneurons. Changes to normal OPA1 expression have also been linked to Alzheimer's disease, Huntington's disease and Parkinson's disease (see Wang et al. (2009) J. Neurosci. 29(28): 9090-9103; Costa et al. (2010) EMBO Mol. Med. 2(12): 490-503; Santos et al. (2015) Mol. Neurobiol. 52(1): 573-86; Ramonet et al. (2013) Cell Death Diff. 20(1): 77-85; Iannielli et al. (2018) Cell Rep. 22(8): 2066-2079; and Iannielli et al. (2019) Cell Rep. 29(13): 4646-4656). In one embodiment, the neurological disease is selected from: Alzheimer's disease, Huntington's disease and Parkinson's disease.

In one embodiment, the disease-associated with mitochondrial defects is a prion disease. For example, Wu et al. (2019) Cell Death Dis. 10(10): 710 describe that downregulation of OPA1 is observed in prion disease models in vitro and in vivo, and this occurs concomitantly to mitochondria structure damage and dysfunction, loss of mtDNA, and neuronal apoptosis. These symptoms were alleviated by increasing OPA1 expression.

### Methods

According to a further aspect of the invention, there is provided a method for enhancing protein translation of OPA1 mRNA in a cell comprising administering the functional nucleic acid molecule, DNA molecule, expression vector or composition as defined herein to the cell. Preferably the cell is a mammalian cell, such as a human or a mouse cell.

According to a further aspect of the invention, there is provided a method for increasing the protein synthesis efficiency of OPA1 protein in a cell comprising administering the functional nucleic acid molecule, DNA molecule, expression vector or composition as defined herein to the cell.

The methods described herein may comprise transfecting into a cell the functional nucleic acid molecule, DNA molecule, expression vector or composition as defined herein. The functional nucleic acid molecule, DNA molecule, expression vector or composition may be administered to target cells using methods known in the art and including, for example, microinjection, lipofection, electroporation, using calcium phosphate, self-infection by the vector or transduction of a virus.

In one embodiment, the cell is OPA1 haploinsufficient, i.e, wherein the presence of a variant allele in a heterozygous combination results in the amount of product generated by the single wild-type gene is not sufficient for complete or normal function. Generally, haploinsufficiency is a condition that arises when the normal phenotype requires the protein product of both alleles, and reduction to 50% or less of gene function results in an abnormal phenotype.

Methods of the invention result in increased levels of OPA1 protein in a cell and therefore find use, for example, in methods of treatment for diseases which are associated with OPA1 defects (i.e. reduced OPA1 protein levels and/or loss-of-function mutations of the OPA1 gene). Methods of the invention find particular use in diseases caused by a quantitative decrease in the predetermined, normal protein level. Methods of the invention can be performed in vitro, ex vivo or in vivo.

According to a further aspect of the invention, there is provided a method of treating a disease associated with mitochondrial defects, such as ADOA, comprising administering a therapeutically effective amount of the functional nucleic acid molecule, DNA molecule, expression vector or composition as defined herein.

Gene therapy in diseases such as ADOA is challenging because the double layered IMM is a relatively impermeable barrier. Although allotropic approaches that engineer the protein to be expressed to contain a specific mitochondrial targeting sequence, are currently being investigated, the OPA1 functional nucleic acid molecules as described herein mitigate the need for such a design around, eliciting their effects in the cytoplasm to boost translation of the endogenous target.

In one embodiment the therapeutically effective amount is administered in the retina, brain, or heart, in particular the retina.

It will be understood that the embodiments described herein may be applied to all aspects of the invention, i.e. the embodiment described for the functional nucleic acid molecules may equally apply to the claimed methods and so forth.

The invention will now be illustrated with reference to the following non-limiting examples.

### EXAMPLES

### Example 1

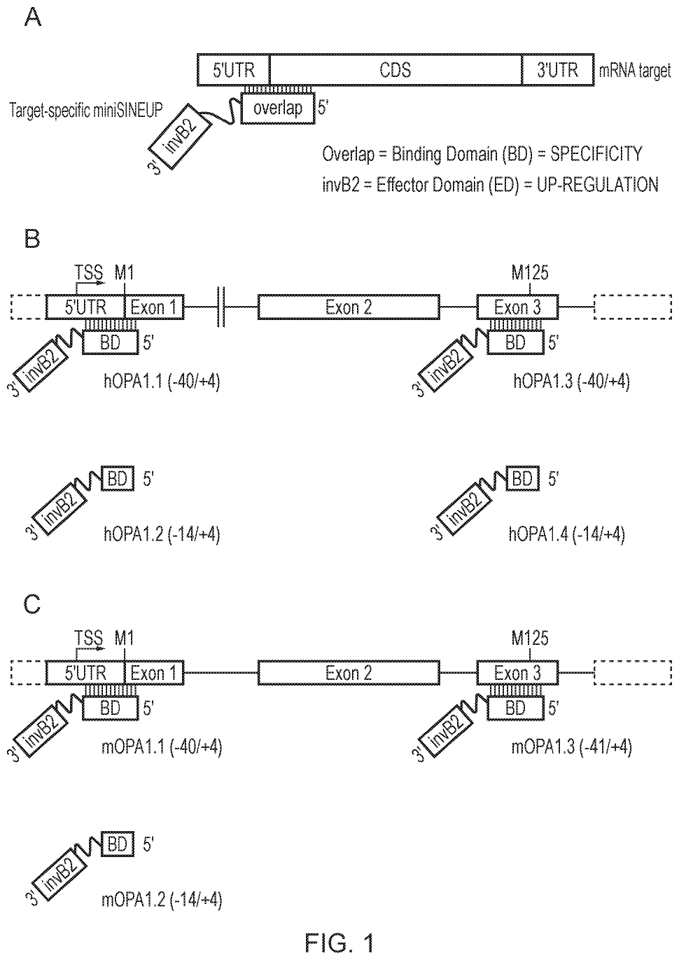

Synthetic miniSINEUPs were designed to target human OPA1 mRNA. FIG. 1A shows a schematic representation of SINEUPs functional domains. The overlap is the Binding Domain (BD, grey) that provides SINEUP specificity and is in antisense orientation to the sense protein-coding mRNA (Target mRNA). The inverted SINE B2 (invB2) element from AS Uchl1 is the Effector Domain (ED) and confers enhancement of protein synthesis. 5′ to 3′ orientation of sense and antisense RNA molecules is indicated. Structural elements of target mRNA are shown: 5′ untranslated region (5′UTR, white), coding sequence (CDS, black) and 3′ untranslated region (3′UTR, white). Scheme is not drawn in scale. (B) Scheme of human OPA1 gene (5′-UTR, white) and BDs (grey) design of synthetic miniSINEUP-OPA1 targeting the initiating M1-AUG and the second in frame M125-AUG. The numbering refers to the position according to the methionine (i.e. −40/+4, from 40 nucleotides upstream and to 4 nucleotides downstream the M1-AUG). All BDs are designed in a region that is included in all human OPA1 transcripts. Scheme is not drawn in scale. (C) Scheme of mouse OPA1 gene (5′-UTR, white) and BDs (grey) design of synthetic miniSINEUP-OPA1 targeting the initiating M1-AUG and the M125-AUG. The numbering refers to the position according to the methionine (i.e. −40/+4, from 40 nucleotides upstream and to 4 nucleotides downstream the M1-AUG). All BDs are designed in a region that is included in all murine OPA1 transcripts. Scheme is not drawn in scale.

### Example 2

This example shows that synthetic miniSINEUPs increase endogenous OPA1 protein level in human cells in vitro. HEK 293T cells were obtained from ATCC (Cat. No. CRL-11268), transfected with miniSINEUPs deprived of BD (ΔBD) and miniSINEUP-OPA1 variants encoded on pCS2+link plasmids and harvested 48 hours post transfection. ΔBD was taken as negative control.

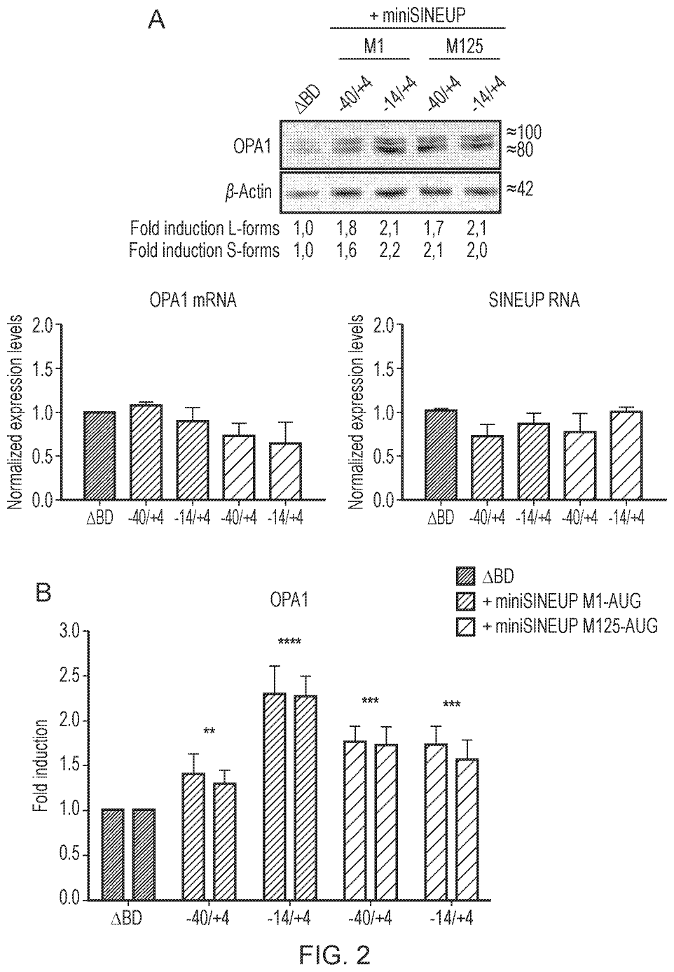

Whole cell lysates were analysed by western blotting with anti-OPA1 and anti-β-actin antibodies. First, OPA1 band (L-forms and S-forms) intensity was normalized to the relative β-actin band. Then, fold change values were calculated normalizing to control cells (ΔBD). Results are shown in FIG. 2A. MiniSINEUP-OPA1-transfected cells show increased levels of endogenous OPA1 protein. Variation in both target and miniSINEUP mRNA expression among samples are not statistically significant (one-way ANOVA followed by Dunnett's post-test). OPA1 transcripts were quantified, using human GAPDH (hGAPDH) expression as an internal control. The OPA1/hGAPDH ratio for the ΔBD sample was set as a baseline value to which all transcripts levels were normalized. Unchanged OPA1 mRNA levels are shown, thereby confirming OPA1 increased protein synthesis at post-transcriptional level. miniSINEUP transcripts were quantified, using hGAPDH expression as internal control. The ΔBD/hGAPDH ratio sample was set as a baseline value to which all transcripts levels were normalized.

FIG. 2B shows average fold change of OPA1 protein levels. All miniSINEUPs are shown to increase endogenous OPA1 protein level.

### Example 3

This example shows that synthetic miniSINEUPs increase endogenous OPA1 protein level in mouse Neuro2A cell line in vitro. Neuro2A (N2A) cells were obtained from ATCC (Cat. No. CCL-131), transfected with miniSINEUP deprived of BD (ΔBD) and miniSINEUP-OPA1 variants encoded on pCS2+link plasmids and harvested 48 hours post transfection. ΔBD was taken as negative control.

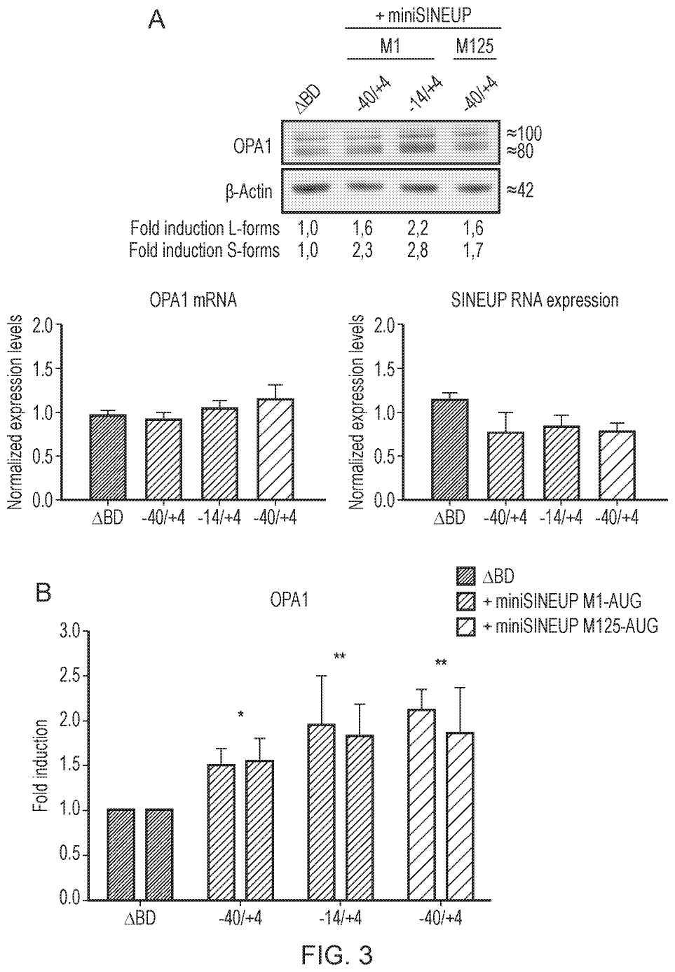

As described hereinbefore, whole cell lysates were analysed by western blotting with anti-OPA1 (BD Bioscience, Cat 612606) and anti-β-actin antibodies (Sigma, Cat. No. A2066). OPA1 band (L-forms and S-forms) intensity was normalized to the relative β-actin band and then fold change values were calculated normalizing to control cells (ΔBD). Results are shown in FIG. 3A. MiniSINEUP-OPA1-transfected cells show increased levels of endogenous OPA1 protein. Variation in both target and miniSINEUP mRNA expression among samples are not statistically significant (one-way ANOVA followed by Dunnett's post-test). OPA1 transcripts were quantified, using mouse GAPDH (mGAPDH) expression as internal control. The OPA1/mGAPDH ratio for ΔBD sample was set as a baseline value to which all transcripts levels were normalized. Unchanged OPA1 mRNA levels are shown, thereby confirming OPA1 increased protein synthesis at post-transcriptional level. miniSINEUP transcripts were quantified, using mGAPDH expression as internal control. The ΔBD/mGAPDH ratio sample was set as a baseline value to which all transcripts levels were normalized.

FIG. 3B shows average fold change of OPA1 protein levels. All miniSINEUPs are shown to increase endogenous OPA1 protein level.

### Example 4

This example shows that synthetic miniSINEUPs increase endogenous OPA1 protein level in mouse Astrocytes cell line in vitro. Astrocyte cells were obtained from ATCC (CRL-254), transfected with miniSINEUP deprived of BD (ΔBD) and miniSINEUP-OPA1 variants encoded on pCS2+link plasmids and harvested 48 hours post transfection. ΔBD was taken as negative control.

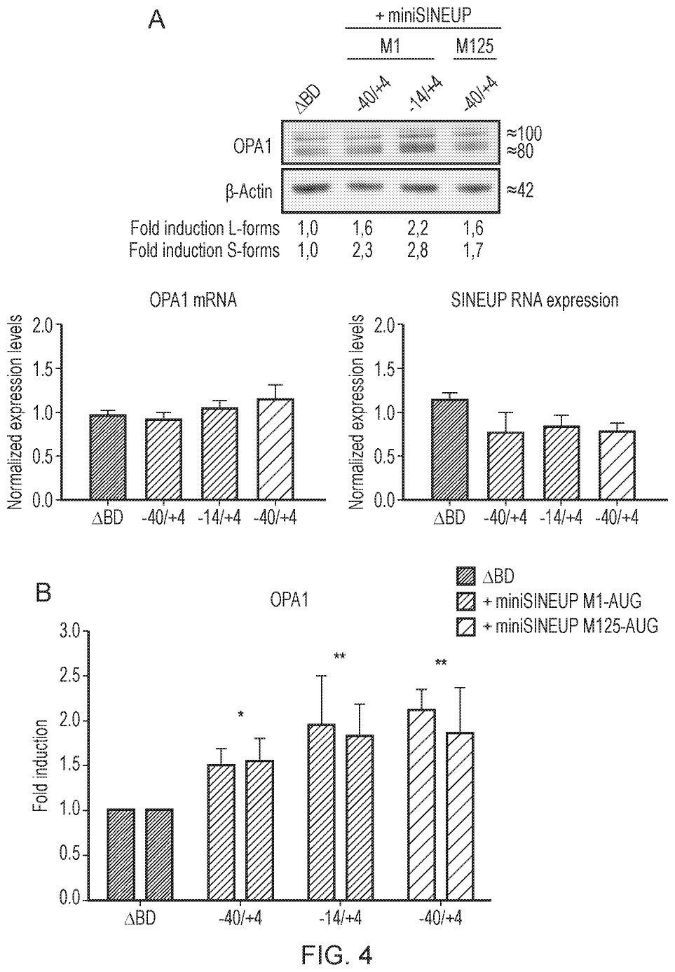

As described hereinbefore, whole cell lysates were analysed by western blotting with anti-OPA1 and anti-β-actin antibodies. OPA1 band (L-forms and S-forms) intensity was normalized to the relative β-actin band and fold change values were calculated normalizing to control cells (ΔBD). Results are shown in FIG. 4A. MiniSINEUP-OPA1-transfected cells show increased levels of endogenous OPA1 protein. Variation in both target and miniSINEUP mRNA expression among samples are not statistically significant (one-way ANOVA followed by Dunnett's post-test). OPA1 transcripts were quantified, using mGAPDH expression as internal control. The OPA1/mGAPDH ratio for ΔBD sample was set as a baseline value to which all transcripts levels were normalized. Unchanged OPA1 mRNA levels are shown, thereby confirming OPA1 increased protein synthesis at post-transcriptional level. miniSINEUP transcripts were quantified, using mGAPDH expression as internal control. The ΔBD/mGAPDH ratio sample was set as a baseline value to which all transcripts levels were normalized.

FIG. 4B shows average fold change of OPA1 protein levels. All miniSINEUPs are shown to increase endogenous OPA1 protein level.

### Example 5

This example describes effector domain (i.e. regulatory sequence) optimization. microSINEUPs increase endogenous OPA1 protein level in HEK 293T cells in vitro. HEK 293T cells were transfected with control vector (ΔBD), miniSINEUP-OPA1 (14/+4-M1-AUG) and microSINEUP-OPA1 (14/+4-M1-AUG) variant. Cells were harvested 48 hours post transfection. Control vector (ΔBD) and miniSINEUP-OPA1 (14/+4-M1-AUG) were taken as negative control and positive controls respectively. MicroSINEUP-OPA1 presents a truncated ED composed by nucleotides 44-120 of the invSINEB2 element from AS Uchl1.

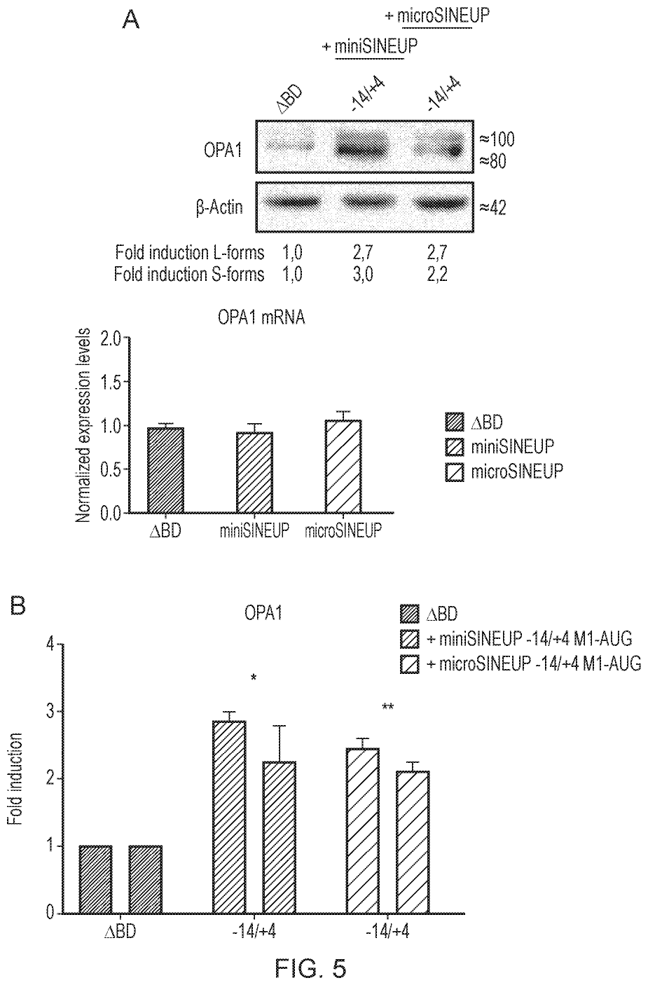

Whole cell lysates were analysed by western blotting with anti-OPA1 and anti-β-actin antibodies. As described hereinbefore, OPA1 band intensity was normalized to the relative β-actin and fold change values were calculated normalizing to negative control cells (ΔBD). Results are shown in FIG. 5. MicroSINEUP-OPA1-transfected cells show increased levels of endogenous OPA1 protein compared to negative control cells.

### Example 6

This example shows that synthetic nano2SINEUPs increase endogenous OPA1 protein level in human cells in vitro. HEK 293T cells were transfected with miniSINEUP lacking a binding domain (ΔBD) and nano2SINEUP-OPA1 (−14/+4-M1-AUG) and harvested 48 hours post transfection. Nano2SINEUP-OPA1 presents a truncated ED composed by nucleotides 64-92 of the invSINEB2 element from AS Uchl1.

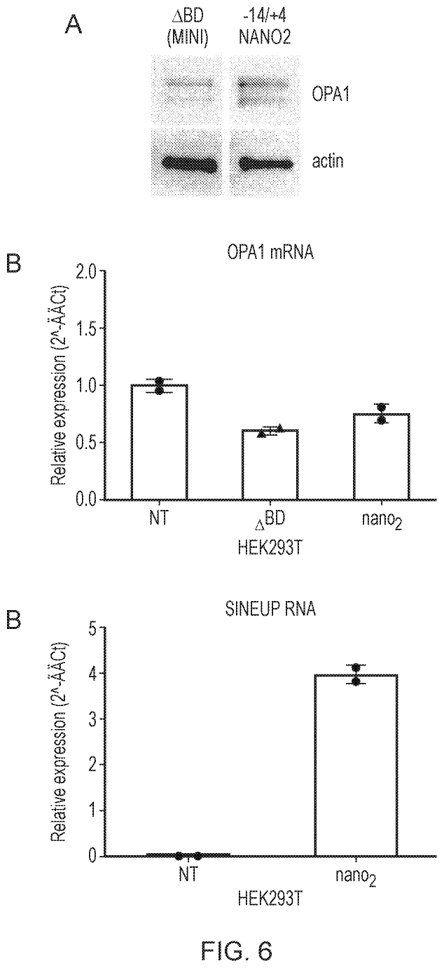

Whole cell lysates were analysed by western blotting with anti-OPA1 and anti-β-actin antibodies. A clear increase in OPA1 protein levels is demonstrated following transfection with nano2SINEUP, but not control SINEUP lacking the BD (FIG. 6A). Real-time PCR analysis of OPA1 mRNA and nano2SINEUP RNA expression in transfected cells shows that endogenous OPA1 mRNA is not significantly increased compared to non-transfected (NT) control cells, confirming that increased OPA1 protein levels occur post-transcriptionally (FIG. 6B, left panel). Real-time PCR analysis also clearly demonstrates the expression and presence of OPA1-nano2SINEUP in HEK 293T cells (FIG. 6B, right panel).

### Example 7

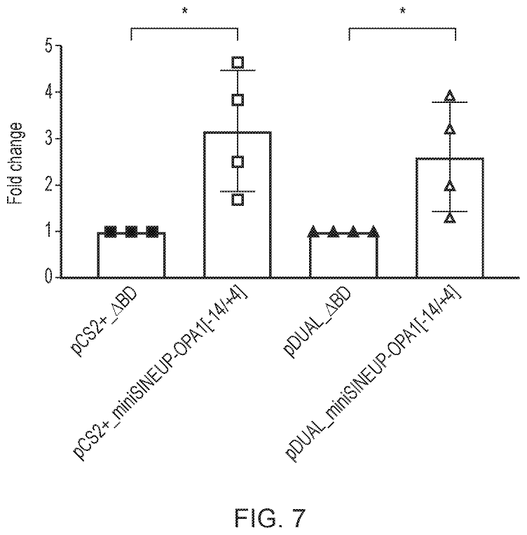

This example shows miniSINEUPs in two different vector backbones increase overexpressed OPA1-nanoluc luminescence in vitro. Mouse Neuro2A cells were co-transfected with an expression plasmid containing nanoluciferase-tagged human OPA1, plus either miniSINEUP lacking a binding domain (ΔBD) or miniSINEUP-OPA1 (with −14/+4 binding domain encompassing Methionine 1). Two different plasmid backbones were used, pCS2+ and pDUAL. After 48 hours, cells were subjected to luciferase assay to quantify the amount of luminescence present in each of the treatment conditions. For both plasmid vector backbones, those containing the miniSINEUP-OPA1 showed ˜2.5 to 3-fold increased luminescence over the negative control lacking the binding domain for the target mRNA (FIG. 7). The pCS2+miniSINEUP-OPA1 showed a 3.2-fold increase over control and the pDUAL was 2.6-fold over control (n=4 independent biological experiments, each with three technical replicates).

### Example 8

This example shows that synthetic nano2SINEUPs increase endogenous OPA1 protein level in human cells in vitro both when transfected in a plasmid vector (pCS2+) and endogenously transcribed and when transfected as naked RNA carrying modified ribonucleotides. The naked RNA molecules were modified using 2′-O-Methyladenosine (2′-O-MeA).

HEK 293T cells were transfected with miniSINEUP lacking a binding domain (ΔBD), nano2SINEUP-OPA1 (−14/+4-M1-AUG), nano2SINEUP lacking a binding domain 2′-O-MeA modified RNA and nano2SINEUP-OPA1 (−14/+4-M1-AUG) 2′-O-MeA modified RNA. Cells were harvested 48 hours post transfection. Nano2SINEUP-OPA1 presents a truncated ED composed by nucleotides 64-92 of the invSINEB2 element from AS Uchl1.

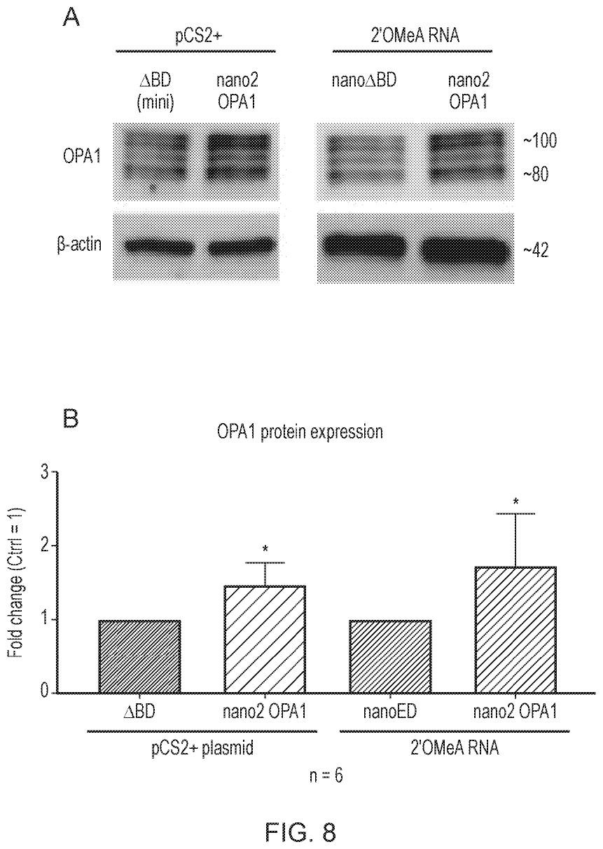

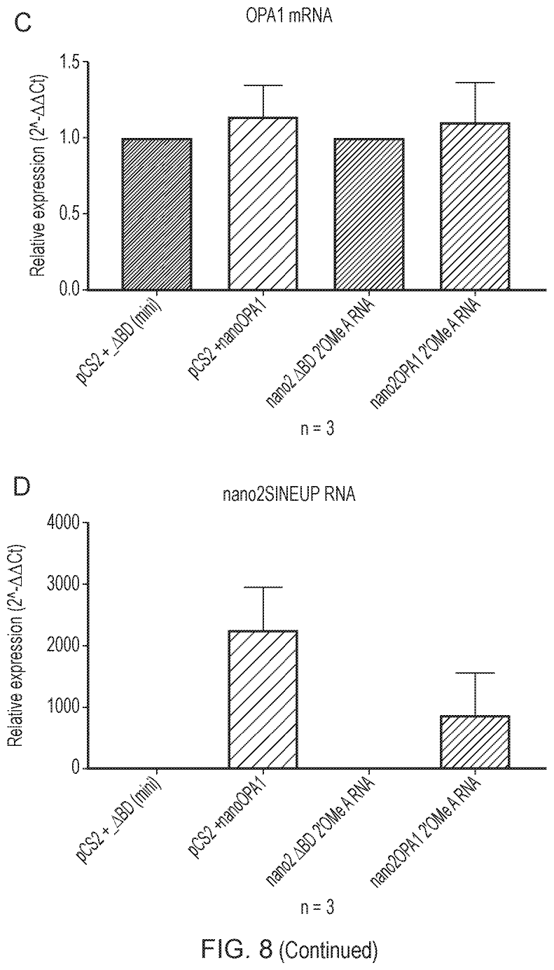

Whole cell lysates were analysed by Western blotting with anti-OPA1 and anti-β-actin antibodies (FIG. 8A). A clear increase in OPA1 protein levels is demonstrated following transfection with nano2SINEUPs (in both plasmid and naked RNA form), but not control SINEUPs lacking the BD (FIGS. 8A and 8B). Real-time PCR analysis of OPA1 mRNA and nano2SINEUP RNA expression in transfected cells shows that endogenous OPA1 mRNA is not significantly increased compared to control cells, confirming that increased OPA1 protein levels occur post-transcriptionally (FIG. 8C). Real-time PCR analysis also clearly demonstrates the expression and presence of OPA1-nano2SINEUP in HEK 293T cells (FIG. 8D, right panel).

1.-20. (canceled)

21. A functional nucleic acid molecule comprising:
at least one target binding sequence comprising a sequence reverse complementary to an OPA1 mRNA sequence; and
at least one regulatory sequence comprising an RNA comprising a SINE B2 element or a functionally active fragment of a SINE B2 element.

22. The functional nucleic acid molecule according to claim 21, wherein the at least one regulatory sequence comprises a sequence with at least 75% sequence identity with a sequence selected from the group consisting of SEQ ID NO: 1-51.

23. The functional nucleic acid molecule according to claim 21, wherein the at least one regulatory sequence comprises a sequence with at least 90% sequence identity with a sequence selected from the group consisting of SEQ ID NO: 1-51.

24. The functional nucleic acid molecule according to claim 21, wherein the at least one target binding sequence comprises a sequence reverse complementary to a portion of the OPA1 mRNA sequence that is common to all OPA1 isoforms.

25. The functional nucleic acid molecule according to claim 21, wherein the at least one target binding sequence is at least 10 nucleotides long and comprises, from 3′ to 5′:
a sequence reverse complementary to 0 to 50 nucleotides of the 5′ untranslated region (5′ UTR) and 0 to 40 nucleotides of the coding sequence (CDS) of the OPA1 mRNA sequence; or
a sequence reverse complementary to 0 to 80 nucleotides of the region upstream of an AUG site (start codon) of the OPA1 mRNA and 0 to 40 nucleotides of the CDS of the OPA1 mRNA sequence downstream of said AUG site.

26. The functional nucleic acid molecule according to claim 21, wherein the at least one target binding sequence is at least 14 nucleotides long and comprises, from 3′ to 5′:
a sequence reverse complementary to 0 to 40 nucleotides of the 5′ UTR and 0 to 32 nucleotides of the CDS of the OPA1 mRNA sequence; or
a sequence reverse complementary to 0 to 70 nucleotides of the region upstream of an AUG site (start codon) of the OPA1 mRNA and 0 to 4 nucleotides of the CDS of the OPA1 mRNA sequence downstream of said AUG site.

27. The functional nucleic acid molecule according to claim 21, further comprising at least one linker sequence between the at least one target binding sequence and the at least one regulatory sequence.

28. The functional nucleic acid molecule according to claim 21, wherein the molecule is circular.

29. A DNA molecule encoding the functional nucleic acid molecule according to claim 21.

30. An expression vector comprising (a) the functional nucleic acid molecule according to claim 21 or (b) a DNA molecule encoding the functional nucleic acid molecule according to claim 21.

31. A composition comprising (a) the functional nucleic acid molecule according to claim 21, (b) a DNA molecule encoding the functional nucleic acid molecule according to claim 21, or (c) an expression vector comprising (a) or (b).

32. A method for increasing the protein synthesis efficiency of OPA1 in a cell comprising administering to the cell (a) the functional nucleic acid molecule according to claim 21, (b) a DNA molecule encoding the functional nucleic acid molecule according to claim 21, (c) an expression vector comprising (a) or (b), or (d) a composition comprising (a), (b), or (c).

33. The method according to claim 32, wherein the functional nucleic acid molecule is administered as naked RNA.

34. The method according to claim 32, wherein the cell is OPA1 haploinsufficient.

35. A method of treating a disease associated with mitochondrial defects comprising administering a therapeutically effective amount of (a) the functional nucleic acid molecule according to claim 21, (b) a DNA molecule encoding the functional nucleic acid molecule according to claim 21, (c) an expression vector comprising (a) or (b), or (d) a composition comprising (a), (b), or (c).

36. The method according to claim 35, wherein the therapeutically effective amount is administered in the retina, brain, or heart.

37. The method according to claim 35, wherein the disease is a neurological disease or a prion disease.

38. The method according to claim 35, wherein the disease is ADOA, Alzheimer's disease, Huntington's disease, or Parkinson's disease.

39. The method according to claim 35, wherein the disease is ADOA.


SEQUENCE LISTING (96 sequences)

SEQ ID NO: 1 - RNA, 167 nt, Mus musculus
cagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcucc

SEQ ID NO: 2 - RNA, 77 nt, Mus musculus
gaacuggaguuauacgguaaccucguggugguugugaaccaccauguggauggauauugaguuccaaacacuggucc

SEQ ID NO: 3 - RNA, 29 nt, Mus musculus
ccucguggugguugugaaccaccaugugg

SEQ ID NO: 4 - RNA, 61 nt, Mus musculus
guuauacgguaaccucguggugguugugaaccaccauguggauggauauugaguuccaaac

SEQ ID NO: 5 - RNA, 98 nt, Mus musculus
aucccccagaacuggaguuauacgguaaccucguggugguugugaaccaccauguggauggauauugaguuccaaacacugguccugugcaagagcau

SEQ ID NO: 6 - RNA, 129 nt, Mus musculus
gaagagggcauuggaucccccagaacuggaguuauacgguaaccucguggugguugugaaccaccauguggauggauauugaguuccaaacacugguccugugcaagagcauccagugcucuuaagugc

SEQ ID NO: 7 - RNA, 183 nt, Mus musculus
gggcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaagcu

SEQ ID NO: 8 - RNA, 67 nt, Mus musculus
gaacuggaguuauacgguaaccucguggugguugugaaccaccauguggauggauauugaguuccaa

SEQ ID NO: 9 - RNA, 107 nt, Mus musculus
ggaucccccagaacuggaguuauacgguaaccucguggugguugugaaccaccauguggauggauauugaguuccaaacacugguccugugcaagagcauccagugc

SEQ ID NO: 10 - RNA, 127 nt, Mus musculus
agagggcauuggaucccccagaacuggaguuauacgguaaccucguggugguugugaaccaccauguggauggauauugaguuccaaacacugguccugugcaagagcauccagugcucuuaagugc

SEQ ID NO: 11 - RNA, 38 nt, Mus musculus
gguaaccucguggugguugugaaccaccauguggaugg

SEQ ID NO: 12 - RNA, 180 nt, Artificial sequence
  mutated functionally active fragment of inverted SINE B2 transposable element derived from AS Uchl 1
cagugcuagaggaggucagaagagggcauuggaucccccagaacuggaguuauacgauaaccucguggugguugugaaccaccauguggauggauauugaguuccaaacacugguccugugcaagagcauccagugcucuuaagugcugagccaucucuuuagcuccagucucuuaagcu

SEQ ID NO: 13 - RNA, 180 nt, Artificial sequence
  mutated functionally active fragment of inverted SINE B2 transposable element derived from AS Uchl 1
cagugcuagaggaggucagaagagggcauuggaucccccagaacuggaguuauacgcuaaccucguggugguugugaaccaccauguggauggauauugaguuccaaacacugguccugugcaagagcauccagugcucuuaagugcugagccaucucuuuagcuccagucucuuaagcu

SEQ ID NO: 14 - RNA, 180 nt, Artificial sequence
  mutated functionally active fragment of inverted SINE B2 transposable element derived from AS Uchl 1
cagugcuagaggaggucagaagagggcauuggaucccccagaacuggcguuauacgguaaccucguggugguugugaaccaccauguggauggauauugaguuccaaacacugguccugugcaagagcauccagugcucuuaagugcugagccaucucuuuagcuccagucucuuaagcu

SEQ ID NO: 15 - RNA, 180 nt, Artificial sequence
  mutated functionally active fragment of inverted SINE B2 transposable element derived from AS Uchl 1
cagugcuagaggaggucagaagagggcauuggaucccccagaaguggaguuauacgguaaccucguggugguugugaaccaccauguggauggauauugaguuccaaacacugcuccugugcaagagcauccagugcucuuaagugcugagccaucucuuuagcuccagucucuuaagcu

SEQ ID NO: 16 - RNA, 180 nt, Artificial sequence
  mutated functionally active fragment of inverted SINE B2 transposable element derived from AS Uchl 1
cagugcuagaggaggucagaagagggcauuggaucccccagauggugaguuauacgguaaccucguggugguugugaaccaccauguggauggauauugaguuccaaacacgucaccugugcaagagcauccagugcucuuaagugcugagccaucucuuuagcuccagucucuuaagcu

SEQ ID NO: 17 - RNA, 180 nt, Artificial sequence
  mutated functionally active fragment of inverted SINE B2 transposable element derived from AS Uchl 1
cagugcuagaggaggucagaagagggcauuggaucccccagaacugcacuauacgguaaccucguggugguugugaaccaccauguggauggauauugaguuccaaaugagugguccugugcaagagcauccagugcucuuaagugcugagccaucucuuuagcuccagucucuuaagcu

SEQ ID NO: 18 - RNA, 116 nt, Artificial sequence
  functionally active fragment of inverted SINE B2 transposable element derived from AS Uchl1
gggcauuggaucccccagaacuggaguuauacgguaaccucguggugguugugaaccaccauguggauggauauugaguuccaaacacugguccugugcaagagcauccagugcuc

SEQ ID NO: 19 - RNA, 77 nt, Artificial sequence
  mutated functionally active fragment of inverted SINE B2 transposable element derived from AS Uchl 1
ggacuggaguuauacgguaaccucguggugguugugaaccaccauguggauggauauugaguuccaaacacuggucc

SEQ ID NO: 20 - RNA, 77 nt, Artificial sequence
  mutated functionally active fragment of inverted SINE B2 transposable element derived from AS Uchl 1
gaacuggcguuauacgguaaccucguggugguugugaaccaccauguggauggauauugaguuccaaacacuggucc

SEQ ID NO: 21 - RNA, 107 nt, Mus musculus
gaugccuuagaaguggaguuaagaguugugagcugccguuuuuugguucugggacucgaacucguuuccucugauacuaucaaccaccaagccaucucuucagcccc

SEQ ID NO: 22 - RNA, 131 nt, Mus musculus
gccagaagaaguugugggauucccuggaacuggagcaaccaacaguuugugugcaccauguggguaaugggaaucgaaccuggguccucuauaagacuggccagugcucuuaacuacugaggugcauuucu

SEQ ID NO: 23 - RNA, 187 nt, Mus musculus
uuauuuuaaauauaugaguauuucaccugcauaggcgcacaguacccacagagacuagaagaggguggcagaucuccugagacuggaguuaaugcuugugagcugccauguggaugcuggaaaucaaacccagguccuuuggaaggcaggcaggugcucuuaaucauggaagcaucucuucagcucc

SEQ ID NO: 24 - RNA, 131 nt, Mus musculus
cagcgacaucagaagaggauauuggaucccauuacagaugguugaaggccaccaugucguugcugggaaugaacucaagaccucuggaagagcagucagugcucuuaaccucugagccaucucuccagccc

SEQ ID NO: 25 - RNA, 114 nt, Mus musculus
auccccuccaaagcucaagaugguuguaagccacccugugauugcugggauuugaacucaagaccuccggaagagcaauuagugcucuuaaccgcugagcaaucucuccagccc

SEQ ID NO: 26 - RNA, 357 nt, Mus musculus
gugcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccuuauuuuaaauauaugaguauuucaccugcauaggcgcacaguacccacagagacuagaagaggguggcagaucuccugagacuggaguuaaugcuugugagcugccauguggaugcuggaaaucaaacccagguccuuuggaaggcaggcaggugcucuuaaucauggaagcaucucuucagcucc

SEQ ID NO: 27 - RNA, 532 nt, Mus musculus
gugcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccgugcgaauucggugcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccgugcgaauucggugcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcucc

SEQ ID NO: 28 - RNA, 228 nt, Mus musculus
uuuuuuuaaaaauuuauuuuuauuuuauguguaugaguguuuugccugcauguaugucuguguaccacgugcgugccuggugcccgcggaggccagaagagggcgucggauccccuggaacuggaguuacagaugguugugagccgccaugugggugcugggaaucgaacccggguccucuggaagagcagccagugcucuuaaccgcugagccaucucuccagcccc

SEQ ID NO: 29 - RNA, 214 nt, Mus musculus
uuuuuuuuacuuguauagguguuuugccugcauguguaucuaucuauguaccgaauauguuccugguauccacagagaccaaaaguggauguuguaucuccugaaauuggagucauagacaguuaugagcugccauuugagugcuuggaauagaacccagguccucuuaaagagcauccagugcucuuaaaaacugagacaucucuguagccuc

SEQ ID NO: 30 - RNA, 200 nt, Mus musculus
uuuauuuugcuuuaugugucugaguguuugcuugaauguaugucuguguaccacgccuguaccuugugccuucagaguugagaggagggcauaggaucuccuggaacuggaauugcaggugguugugagccacccuguggguccuggggaccauacuccagcaagaacaucaugugcucuuaauuccugagucuccaacc

SEQ ID NO: 31 - RNA, 214 nt, Mus musculus
uuuauuuacuuaucuuuauguguaugaguguguugucagacuguuaugucugugugucacaugcaugccugcuguucauggaguccagaagagggcaucggauccccuggaacuggaguuacagaugaguggccaugugaauguuaagaaccaaaccuggguccucugaaagagcagacaaugcucuuaacuacugagcugucucuccagcccc

SEQ ID NO: 32 - RNA, 205 nt, Mus musculus
uuauuuuauucguguaaguguuuugccagcaucuaugucuucgcacuaugugcaggucuggugccugagggguccagacgagagcacugggucuccgggaacuggaguuacagaucauugugagccaccaugugggugcagggaaucgaaccugggaccucuggaggagcagccacugcucuuaaccacuacacuauuucuccag

SEQ ID NO: 33 - RNA, 121 nt, Mus musculus
ucuguggaccacuguguacagaagccugagaaggcuagcagauccccagaacuggaacugugagacgcugugcuauggaggugcuaggaacugaaaauggauggguccucugcaagagcag

SEQ ID NO: 34 - RNA, 191 nt, Mus musculus
uuguuuuaauugaauggcuauaggguguuucuucuguauguauaucuauguuugguaccuacagaggcaucagauccucuggaacuguaguugcugacaguugugagcugucauggggaugcuggaauugaaccuggauccuaugaaagaacagccaguguucuuaaccgcugagcuaucucuccaggccc

SEQ ID NO: 35 - RNA, 205 nt, Mus musculus
uuuuuuuuuuaauuuuaaaaaaaaagauuuuauuuauuuauuuuauauaugaugaguacacugucacucuuuucagacacccuagaaaaggggggcaucagaucccauuacagaugguugugagccacaugguugcugggaauugaccucaggaccucugaaagagcagucagugcucucaaccuuugagucaucucuccagccc

SEQ ID NO: 36 - RNA, 190 nt, Mus musculus
auguauaucuguaaugggacauacucacauacaugggcacgugaguauaaaaggccagaagagagcacuggacccucuggaguugagauucuaagcaguugugaaccaucugauguaggugcugggaacugaacuuggguccuuugcuagagaaguaugucucuuaaccacugagccguaucuccauccc

SEQ ID NO: 37 - RNA, 169 nt, Mus musculus
uaaagauuuauucauuaaguacacuguagcuaucuucagacgcaucagaagagggcgucagaucucuuuacaggugguugugagccaccaugugguugcuggaauuugaacucaggaccuucaaaagagcagucaguguucuuaaccgcugagccaucucuccaacccc

SEQ ID NO: 38 - RNA, 159 nt, Mus musculus
uuauuuauuauaaguacacuguagcugucuucagacacaacaaaagagggcgucagaucucauuacaggugguugagccaccaugugguugcugggauuugaacucaggaccuucagaacagucagugcucuuacccacugagccagcgagccagcccc

SEQ ID NO: 39 - RNA, 77 nt, Artificial sequence
  mutated functionally active fragment of inverted SINE B2 transposable element derived from AS Uchl 1
gaacuggaguuauacgguaaccucguggugguugggaaccaccauguggauggauauugaguuccaaacacuggucc

SEQ ID NO: 40 - RNA, 77 nt, Artificial sequence
  mutated functionally active fragment of inverted SINE B2 transposable element derived from AS Uchl 1
gaacuggaguuauacgguaaccucguggugguucccaaccaccauguggauggauauugaguuccaaacacuggucc

SEQ ID NO: 41 - RNA, 77 nt, Artificial sequence
  mutated functionally active fragment of inverted SINE B2 transposable element derived from AS Uchl 1
ggaccggaguuauacgguaaccgcguggugguugugaaccaccacgcggauggauauugaguuccaaacaccggucc

SEQ ID NO: 42 - RNA, 77 nt, Artificial sequence
  mutated functionally active fragment of inverted SINE B2 transposable element derived from AS Uchl 1
gaacuagaguuauacgguaaccacauggugguugugaaccaccauguggauggauauugaguuccaaacacuaguuc

SEQ ID NO: 43 - RNA, 206 nt, Artificial sequence
  functionally active fragment of inverted SINE B2 transposable element derived from AS Uchl1
uuauuuuaaauauaugaguauuucaccugcauaggcgcacaguacccacagagacuagaagaggguaguagauccccuagaacuggaguuauacgguaaccucguggugguugugagcuaccauguggauggauacugggaaucaaacccagguccuguggaaggcaggcaggugcucucaagcacugagccaucucuucagcucc

SEQ ID NO: 44 - RNA, 206 nt, Artificial sequence
  functionally active fragment of inverted SINE B2 transposable element derived from AS Uchl1
uuauuuuaaauauaugaguauuucaccugcauaggcgcacagugcucaaggagaucagaagagggcaucagaucuccugagacuggaguuauacgguaaccucgugaugguugugaacuaccauguggauggauauugaguuccaaacacagguccugugcaagagcagcaggugcucuuaagcacggaaccaucucuuuagcucc

SEQ ID NO: 45 - RNA, 156 nt, Artificial sequence
  functionally active fragment of inverted SINE B2 transposable element derived from AS Uchl1
gaggcuagaagaggguaucagauccccugagacuggaguuauacgguaaccucguggugguugugagccaccauguggauggauacugagaaccaaacccugguccugugcaagagcaucaggugcucuuaagcacggaaccaucucuucagcucc

SEQ ID NO: 46 - RNA, 68 nt, Artificial sequence
  functionally active fragment of inverted SINE B2 transposable element derived from AS Uchl1
guccugugcaagagcaucgaacucggugcucuuaagcacagaagccaccaagccaucucuucagcccc

SEQ ID NO: 47 - RNA, 110 nt, Artificial sequence
  functionally active fragment of inverted SINE B2 transposable element derived from AS Uchl1
cagugcuagaggaggucagaagagggcaucccccagccucguggugguugugaaccaccauguggcugugcaagagcaugcucuuaagugcugagccaucucuuuagcuc

SEQ ID NO: 48 - RNA, 126 nt, Artificial sequence
  functionally active fragment of inverted SINE B2 transposable element derived from AS Uchl1
gagggcauuggaucccccagaacuggaguuauacgguaaccucguggugguugugaaccaccauguggauggauauugaguuccaaacacugguccugugcaagagcauccagugcucuuaagugc

SEQ ID NO: 49 - RNA, 89 nt, Artificial sequence
  functionally active fragment of inverted SINE B2 transposable element derived from AS Uchl1
ggaucccccagaacuggaguuauacgguaaccucguggugguugugaaccaccauguggauggauauugaguuccaaacacugguccug

SEQ ID NO: 50 - RNA, 72 nt, Artificial sequence
  functionally active fragment of inverted SINE B2 transposable element derived from AS Uchl1
ugcuagaggaggucagaagagggcauuggaugcaaauccagugcucuuaagugcugagccaucucuuuagcu

SEQ ID NO: 51 - RNA, 94 nt, Artificial sequence
  functionally active fragment of inverted SINE B2 transposable element derived from AS Uchl1
gagggcauuggaucccccagaacuggaguuauacgguaacgauggauauugaguuccaaacacugguccugugcaagagcauccagugcucuua

SEQ ID NO: 52 - RNA, 383 nt, Hepatitis C virus
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguugggucgcgaaaggccuugugguacugccugauagggugcuugcgagugccccgggaggucucguagaccgugcaccaugagcacgaauccuaaaccucaaagaaaaaccaaacguaac

SEQ ID NO: 53 - RNA, 383 nt, Hepatitis C virus
guuacguuugguuuuucuuugagguuuaggauucgugcucauggugcacggucuacgagaccucccggggcacucgcaagcacccuaucaggcaguaccacaaggccuuucgcgacccaacacuacucggcuagcagucuugcgggggcacgcccaaaucuccaggcauugagcggguuuauccaagaaaggacccggucguccuggcaauuccgguguacucaccgguuccgcagaccacuauggcucucccgggaggggggguccuggaggcugcacgacacucauacuaacgccauggcuagacgcuuucugcgugaagacaguaguuccucacaggggagugauucaugguggagugucgcccccaucagggggcuggc

SEQ ID NO: 54 - RNA, 312 nt, Artificial Sequence
  Human poliovirus IRES
augagucuggacaucccucaccggugacggugguccaggcugcguuggcggccuaccuauggcuaacgccaugggacgcuaguugugaacaaggugugaagagccuauugagcuacauaagaauccuccggccccugaaugcggcuaaucccaaccucggagcagguggucacaaaccagugauuggccugucguaacgcgcaaguccguggcggaaccgacuacuuuggguguccguguuuccuuuuauuuuauuguggcugcuuauggugacaaucacagauuguuaucauaaagcgaauuggauuggcc

SEQ ID NO: 55 - RNA, 312 nt, Artificial Sequence
  Human poliovirus IRES
ggccaauccaauucgcuuuaugauaacaaucugugauugucaccauaagcagccacaauaaaauaaaaggaaacacggacacccaaaguagucgguuccgccacggacuugcgcguuacgacaggccaaucacugguuugugaccaccugcuccgagguugggauuagccgcauucaggggccggaggauucuuauguagcucaauaggcucuucacaccuuguucacaacuagcgucccauggcguuagccauagguaggccgccaacgcagccuggaccaccgucaccggugagggauguccagacucau

SEQ ID NO: 56 - RNA, 576 nt, Encephalomyocarditis virus
cccccccucucccuccccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaa

SEQ ID NO: 57 - RNA, 576 nt, Encephalomyocarditis virus
uuaucaucguguuuuucaaaggaaaaccacguccccgugguucggggggccuagacguuuuuuaaccucgacuaaacacauguaaagcaugugcaccgaggccccagaucagaucccauacaaugggguaccuucugggcauccuucagccccuuguugaauacgcuugaggagagccauuugacucuuuccacaacuauccaacucacaacguggcacugggguugugccgccuuugcagguguaucuuauacacguggcuuuuggccgcagaggcaccugucgccaggugggggguuccgcugccugcaaagggucgcuacagacguuguuugucuucaagaagcuuccagaggaacugcuuccuucacgacauucaacagaccuugcauuccuuuggcgagaggggaaagaccccuaggaaugcucgucaagaagacagggccagguuuccgggcccucacauugccaaaagacggcaauaugguggaaaauaacauauagacaaacgcacaccggccuuauuccaagcggcuucggccaguaacguuaggggggggggagggagaggggggg

SEQ ID NO: 58 - RNA, 192 nt, Cricket paralysis virus
aaagcaaaaaugugaucuugcuuguaaauacaauuuugagagguuaauaaauuacaaguagugcuauuuuuguauuuagguuagcuauuuagcuuuacguuccaggaugccuaguggcagccccacaauauccaggaagcccucucugcgguuuuucagauuagguagucgaaaaaccuaagaaauuuaccu

SEQ ID NO: 59 - RNA, 192 nt, Cricket paralysis virus
agguaaauuucuuagguuuuucgacuaccuaaucugaaaaaccgcagagagggcuuccuggauauuguggggcugccacuaggcauccuggaacguaaagcuaaauagcuaaccuaaauacaaaaauagcacuacuuguaauuuauuaaccucucaaaauuguauuuacaagcaagaucacauuuuugcuuu

SEQ ID NO: 60 - RNA, 231 nt, Homo sapiens
cagagauccaggggaggcgccugugaggcccggaccugccccggggcgaaggguauguggcgagacagagcccugcaccccuaauucccgguggaaaacuccuguugccguuucccuccaccggccuggagucucccagucuugucccggcagugccgcccuccccacuaagaccuaggcgcaaaggcuuggcucaugguugacagcucagagagagaaagaucugaggga

SEQ ID NO: 61 - RNA, 231 nt, Homo sapiens
ucccucagaucuuucucucucugagcugucaaccaugagccaagccuuugcgccuaggucuuaguggggagggcggcacugccgggacaagacugggagacuccaggccgguggagggaaacggcaacaggaguuuuccaccgggaauuaggggugcagggcucugucucgccacauacccuucgccccggggcagguccgggccucacaggcgccuccccuggaucucug

SEQ ID NO: 62 - RNA, 460 nt, Homo sapiens
acuuuuggugggcauuuaaaaauguguguguauguguauauauguauguguauguauguguauauauguauauguauguauguaucgcguguauguguguauguaugcauguguauguauguauaugcauguauguguauguguauauauguauguguguguauguauauguguguguauguguauguguguguguauguguguguguauguauguauguauguauauguauuauacacauauacacauauugguuuuuuuaaucauuugagaguuaguugaagauaaaaacccaucaccccuaaauguauuccaaagaauaagaacauuguuuuauacauagcacacuuaacaaaaucaagaaauuuaacauuaauacaguacuguuaccuaauccguagucgauuuucaaauuuugucaguuguuccaauaauguccuuuauauauuccccgcccagc

SEQ ID NO: 63 - RNA, 460 nt, Homo sapiens
gcugggcggggaauauauaaaggacauuauuggaacaacugacaaaauuugaaaaucgacuacggauuagguaacaguacuguauuaauguuaaauuucuugauuuuguuaagugugcuauguauaaaacaauguucuuauucuuuggaauacauuuaggggugauggguuuuuaucuucaacuaacucucaaaugauuaaaaaaaccaauauguguauauguguauaauacauauacauacauacauacauacacacacacauacacacacacauacacauacacacacauauacauacacacacauacauauauacacauacacauacaugcauauacauacauacacaugcauacauacacacauacacgcgauacauacauacauauacauauauacacauacauacacauacauauauacacauacacacacauuuuuaaaugcccaccaaaagu

SEQ ID NO: 64 - RNA, 395 nt, Homo sapiens
aauuccagcgagaggcagagggagcgagcgggcggccggcuaggguggaagagccgggcgagcagagcugcgcugcgggcguccugggaagggagauccggagcgaauagggggcuucgccucuggcccagcccucccgcuugaucccccaggccagcgguccgcaacccuugccgcauccacgaaacuuugcccauagcagcgggcgggcacuuugcacuggaacuuacaacacccgagcaaggacgcgacucucccgacgcggggaggcuauucugcccauuuggggacacuuccccgccgcugccaggacccgcuucucugaaaggcucuccuugcagcugcuuagacgcuggauuuuuuucggguaguggaaaaccagcagccucccgcga

SEQ ID NO: 65 - RNA, 395 nt, Homo sapiens
ucgcgggaggcugcugguuuuccacuacccgaaaaaaauccagcgucuaagcagcugcaaggagagccuuucagagaagcggguccuggcagcggcggggaaguguccccaaaugggcagaauagccuccccgcgucgggagagucgcguccuugcucggguguuguaaguuccagugcaaagugcccgcccgcugcuaugggcaaaguuucguggaugcggcaaggguugcggaccgcuggccugggggaucaagcgggagggcugggccagaggcgaagcccccuauucgcuccggaucucccuucccaggacgcccgcagcgcagcucugcucgcccggcucuuccacccuagccggccgcccgcucgcucccucugccucucgcuggaauu

SEQ ID NO: 66 - RNA, 48 nt, Homo sapiens
gggcacuuugcacuggaacuuacaacacccgagcaaggacgcgacucu

SEQ ID NO: 67 - RNA, 48 nt, Homo sapiens
agagucgcguccuugcucggguguuguaaguuccagugcaaagugccc

SEQ ID NO: 68 - RNA, 71 nt, Homo sapiens
guacugacaucguagauggaaaucauaaacugacucuugguuugauuuggaauauaauccuccacuggcag

SEQ ID NO: 69 - RNA, 71 nt, Homo sapiens
cugccaguggaggauuauauuccaaaucaaaccaagagucaguuuaugauuuccaucuacgaugucaguac

SEQ ID NO: 70 - RNA, 232 nt, Artificial sequence
  functional nucleic acid molecule directed to human OPA1 isoform
acaucccgccggcggggaggucacgcaggcgccgagacggccacaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuu

SEQ ID NO: 71 - RNA, 206 nt, Artificial sequence
  functional nucleic acid molecule directed to human OPA1 isoform
acaucccgccggcggggaaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuu

SEQ ID NO: 72 - RNA, 199 nt, Artificial sequence
  functional nucleic acid molecule directed to human OPA1 isoform
acaucccgccggcggggagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuu

SEQ ID NO: 73 - RNA, 232 nt, Artificial sequence
  functional nucleic acid molecule directed to human OPA1 isoform
ucauaucuuuccacugaucaaaagucuuuuuggcuguguagccaaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuu

SEQ ID NO: 74 - RNA, 206 nt, Artificial sequence
  functional nucleic acid molecule directed to human OPA1 isoform
ucauaucuuuccacugauaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuu

SEQ ID NO: 75 - RNA, 232 nt, Artificial sequence
  functional nucleic acid molecule directed to mouse OPA1 isoform
acaucccgccagagcagaccacacacaggcgcugagacggccacaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuu

SEQ ID NO: 76 - RNA, 206 nt, Artificial sequence
  functional nucleic acid molecule directed to mouse OPA1 isoform
acaucccgccagagcagaaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuu

SEQ ID NO: 77 - RNA, 233 nt, Artificial sequence
  functional nucleic acid molecule directed to mouse OPA1 isoform
ucauaucuuuccauucaucgaagguuuuuuuggcuguauagccacaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuu

SEQ ID NO: 78 - RNA, 114 nt, Artificial sequence
  functional nucleic acid molecule directed to human OPA1 isoform
acaucccgccggcggggaaucugcagaauucgcccuugaacuggaguuauacgguaaccucguggugguugugaaccaccauguggauggauauugaguuccaaacacuggucc

SEQ ID NO: 79 - RNA, 66 nt, Artificial sequence
  functional nucleic acid molecule directed to human OPA1 isoform
acaucccgccggcggggaaucugcagaauucgcccuuccucguggugguugugaaccaccaugugg

SEQ ID NO: 80 - RNA, 13 nt, Artificial sequence
  example of the spacer/linker sequence
aucugcagaauuc

SEQ ID NO: 81 - RNA, 6 nt, Artificial sequence
  example of the spacer/linker sequence
gaauuc

SEQ ID NO: 82 - DNA, 18 bp, Artificial sequence
  -14/+4 of M1
acatcccgccggcgggga

SEQ ID NO: 83 - DNA, 15 bp, Artificial sequence
  -6/+9 of M1
tcgccacatcccgcc

SEQ ID NO: 84 - DNA, 12 bp, Artificial sequence
  -41/+30 of M1
gagacggccacg

SEQ ID NO: 85 - DNA, 14 bp, Artificial sequence
  -97/-87 of M1
aatgacccaggaag

SEQ ID NO: 86 - DNA, 18 bp, Artificial sequence
  -18/-1 of M1
cccgccggcggggaggtc

SEQ ID NO: 87 - DNA, 22 bp, Artificial sequence
  -18/+4 of M1
acatcccgccggcggggaggtc

SEQ ID NO: 88 - DNA, 14 bp, Artificial sequence
  -14/-1 of M1
cccgccggcgggga

SEQ ID NO: 89 - DNA, 17 bp, Artificial sequence
  -9/+8 of M125
ggtatcatatctttcca

SEQ ID NO: 90 - DNA, 18 bp, Artificial sequence
  -18/-1 of M125
atctttccactgatcaaa

SEQ ID NO: 91 - DNA, 22 bp, Artificial sequence
  -18/+4 of M125
tcatatctttccactgatcaaa

SEQ ID NO: 92 - DNA, 14 bp, Artificial sequence
  -14/-1 of M125
atctttccactgat

SEQ ID NO: 93 - DNA, 12 bp, Artificial Sequence
  -41/-30 of M1
gagacggccacg

SEQ ID NO: 94 - DNA, 44 bp, Artificial Sequence
  -40/+4 of M1
acatcccgccggcggggaggtcacgcaggcgccgagacggccac

SEQ ID NO: 95 - DNA, 44 bp, Artificial Sequence
  -40/+4 of M2
tcatatctttccactgatcaaaagtctttttggctgtgtagcca

SEQ ID NO: 96 - DNA, 18 bp, Artificial Sequence
  M125 -14/+4
tcatatctttccactgat